(19) United States
(12) Patent Application Publication
(10) Pub. No.: US 2006/0229271 A1
(43) Pub. Date: Oct. 12, 2006

(54) Methods for treating infectious disease exacerbated asthma

(75) Inventors: Arthur M. Krieg, Wellesley, MA (US); George Tilo De Sanctis, Skillman, NJ (US); Stephen Leslie Underwood, Bedminster, NJ (US); Raymond Anthony Jupp, Chatham, NJ (US); John A. Schmidt JR., Green Brook, NJ (US)

Correspondence Address: WOLF GREENFIELD & SACKS, PC, FEDERAL RESERVE PLAZA, 600 ATLANTIC AVENUE, BOSTON, MA, 02210-2206, US

(73) Assignees: Coley Pharmaceutical Group, Inc., Wellesley, MA (US); Sanofi-Aventis U.S.P. LLC, Bridgewater, NJ (US)

(21) Appl. No.: 11/401,093

(22) Filed: Apr. 10, 2006

Related U.S. Application Data
(60) Provisional application No. 60/669,548, filed on Apr. 8, 2005.

(51) Int. Cl. A61K 48/00

(52) U.S. Cl. 514/44

(57) ABSTRACT

The invention relates to methods of treating infectious disease exacerbated asthma. In particular, the methods are useful for treating viral exacerbated asthma using CpG oligonucleotides.

### RELATED APPLICATION

This application claims the benefit under 35 U.S.C. § 119(e) of U.S. Provisional Application No. 60/669,584, filed Apr. 8, 2005, entitled METHODS FOR TREATING INFECTIOUS DISEASE EXACERBATED ASTHMA, the entire contents of which is hereby incorporated by reference.

### FIELD OF THE INVENTION

The present invention relates generally to methods of treating asthma that is exacerbated by infectious disease using immunostimulatory oligonucleotides, as well as compositions thereof.

### BACKGROUND OF THE INVENTION

Bacterial DNA has immune stimulatory effects to activate B cells and natural killer cells, but vertebrate DNA does not (Tokunaga, T., et al., 1988. Jpn. J. Cancer Res. 79:682-686; Tokunaga, T., et al., 1984, JNCI 72:955-962; Messina, J. P., et al., 1991, J. Immunol. 147:1759-1764; and reviewed in Krieg, 1998, In: Applied Oligonucleotide Technology, C. A. Stein and A. M. Krieg, (Eds.), John Wiley and Sons, Inc., New York, N.Y., pp. 431-448). It is now understood that these immune stimulatory effects of bacterial DNA are a result of the presence of unmethylated CpG dinucleotides in particular base contexts (CpG motifs), which are common in bacterial DNA, but methylated and underrepresented in vertebrate DNA (Krieg et al, 1995 Nature 374:546-549; Krieg, 1999 Biochim. Biophys. Acta 93321:1-10). The immune stimulatory effects of bacterial DNA can be mimicked with synthetic oligodeoxynucleotides (ODN) containing these CpG motifs. Such CpG ODN have highly stimulatory effects on human and murine leukocytes, inducing B cell proliferation; cytokine and immunoglobulin secretion; natural killer (NK) cell lytic activity and IFN-γ secretion; and activation of dendritic cells (DCs) and other antigen presenting cells to express costimulatory molecules and secrete cytokines, especially the Th1-like cytokines that are important in promoting the development of Th1-like T cell responses. These immune stimulatory effects of native phosphodiester backbone CpG ODN are highly CpG specific in that the effects are dramatically reduced if the CpG motif is methylated, changed to a GpC, or otherwise eliminated or altered (Krieg et al, 1995 Nature 374:546-549; Hartmann et al, 1999 Proc. Natl. Acad. Sci USA 96:9305-10).

In early studies, it was thought that the immune stimulatory CpG motif followed the formula purine-purine-CpG-pyrimidine-pyrimidine (Krieg et al, 1995 Nature 374:546-549; Pisetsky, 1996 J. Immunol. 156:421-423; Hacker et al., 1998 EMBO J. 17:6230-6240; Lipford et al, 1998 Trends in Microbiol. 6:496-500). However, it is now clear that mouse lymphocytes respond quite well to phosphodiester CpG motifs that do not follow this “formula” (Yi et al., 1998 J. Immunol. 160:5898-5906) and the same is true of human B cells and dendritic cells (Hartmann et al, 1999 Proc. Natl. Acad. Sci USA 96:9305-10; Liang, 1996 J. Clin. Invest. 98:1119-1129).

Several different classes of CpG oligonucleotides has recently been described. One class is potent for activating B cells but is relatively weak in inducing IFN-α and NK cell activation; this class has been termed the B class. The B class CpG oligonucleotides typically are fully stabilized and include an unmethylated CpG dinucleotide within certain preferred base contexts. See, e.g., U.S. Pat. Nos. 6,194,388; 6,207,646; 6,214,806; 6,218,371; 6,239,116; and 6,339,068. Another class of CpG oligonucleotides activates B cells and NK cells and induces IFN-α; this class has been termed the C-class. The C-class CpG oligonucleotides, as first characterized, typically are fully stabilized, include a B class-type sequence and a GC-rich palindrome or near-palindrome. This class has been described in co-pending U.S. provisional patent application 60/313,273, filed Aug. 17, 2001 and US10/224,523 filed on Aug. 19, 2002 and related PCT Patent Application PCT/US02/26468 published under International Publication Number WO 03/015711.

### SUMMARY OF THE INVENTION

It has been discovered herein that CpG oligonucleotides (CpG ODN) are particularly effective in combating infections, and particularly upper respiratory tract virus, that are a cause of asthma exacerbations. In some aspects of the invention C-class CpG ODN are particularly effective for carrying out the methods. As shown in the Examples below, C-class CpG ODN induced a panel of IFN-associated genes in the mouse, including those for anti-viral proteins, and protected against airway inflammation exacerbated by combined antigen and virus exposures.

In some aspects the invention relates to a method for treating viral exacerbated asthma, by administering to an asthmatic subject an effective amount of a C-class CpG oligonucleotide for treating viral exacerbated asthma.

In other aspects the invention relates to a method for treating viral exacerbated asthma by identifying an asthmatic subject at risk of viral infection, and administering to the asthmatic subject an effective amount of a CpG oligonucleotide for treating viral exacerbated asthma. The subject may be identified by a medical worker. In other embodiments the subject is identified based on exposure to a risk factor for viral infection.

According to other aspects the invention is a method for treating viral exacerbated asthma by administering to an asthmatic subject undergoing a non-CpG asthma therapy an effective amount of a CpG oligonucleotide for treating viral exacerbated asthma. The non-CpG asthma therapy may be a steroid therapy. In some embodiments the non-CpG asthma therapy is administered at a different time than the CpG oligonucleotide. In other embodiments the non-CpG asthma therapy is administered at the same time as the CpG oligonucleotide.

A method for treating infectious disease exacerbated asthma by identifying an asthmatic subject at risk of infection, and administering to the asthmatic subject an effective amount of a CpG oligonucleotide for treating infectious disease exacerbated asthma is provided according to other aspects of the invention.

In another aspect the invention is a method for treating viral exacerbated asthma, by identifying a risk factor for viral infection, and administering to an asthmatic subject an effective amount of a CpG oligonucleotide for treating viral exacerbated asthma during a period of time when the asthmatic subject is at risk of viral infection. In some embodiments the risk factor is influenza season. In other embodiments the risk factor is travel to a destination with a high risk of viral exposure.

In some embodiments the viral exacerbated asthma is caused by a respiratory virus. Optionally the respiratory virus is not RSV. In other embodiments the viral exacerbated asthma is caused by influenza virus.

The CpG oligonucleotide in some embodiments is a C-class oligonucleotide. The C-class oligonucleotide may optionally be a semi-soft oligonucleotide, such as, for instance, SEQ ID NO: 10.

A method for treating viral exacerbated asthma, by identifying an asthmatic subject at risk of viral infection, and administering to the asthmatic subject a CpG oligonucleotide in an amount that is sub-therapeutic for treating viral infection, wherein the CpG oligonucleotide is effective for reducing immune cell accumulation is also provided. The immune cell may be, for instance, a neutrophil or an eosinophil.

In other aspects the invention is a method for treating viral exacerbated asthma, by identifying an asthmatic subject at risk of viral infection, and administering to the asthmatic subject at least three doses of CpG oligonucleotide, wherein the at least three doses of CpG oligonucleotide are temporally separated from one another by at least three days. In some embodiments the doses are separated from one another by 1 week, 2 weeks, 3 weeks, one month, one year or any integer value there between.

Use of an oligonucleotide of the invention for stimulating an immune response and or the treatment of viral exacerbated asthma is also provided as an aspect of the invention.

A method for manufacturing a medicament of an oligonucleotide of the invention for stimulating an immune response and or the treatment of viral exacerbated asthma is also provided.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### BRIEF DESCRIPTION OF DRAWINGS

The figures are illustrative only and are not required for enablement of the invention disclosed herein.

FIG. 1 is a schematic of an abbreviated study schedule showing some of the experimental conditions carried out in Example 1 and 2.

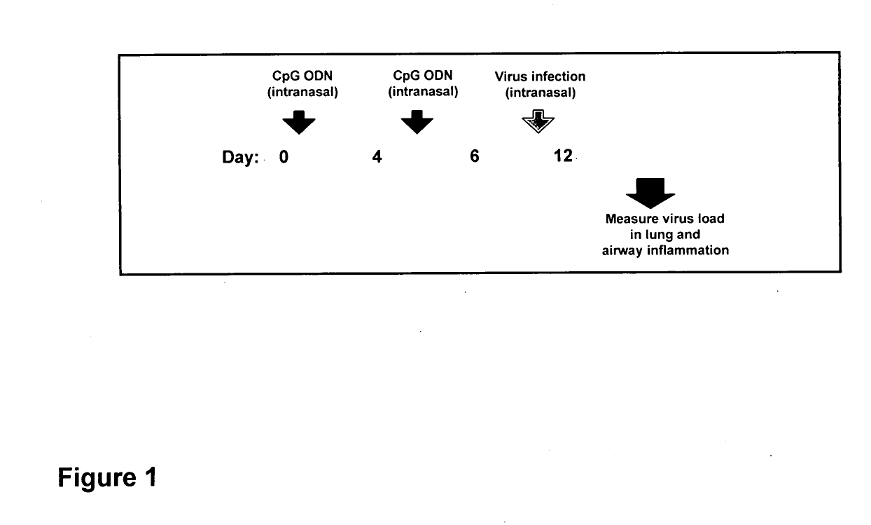

FIG. 2 is a schematic of a detailed study schedule showing an experimental condition carried out in Example 1 (#3).

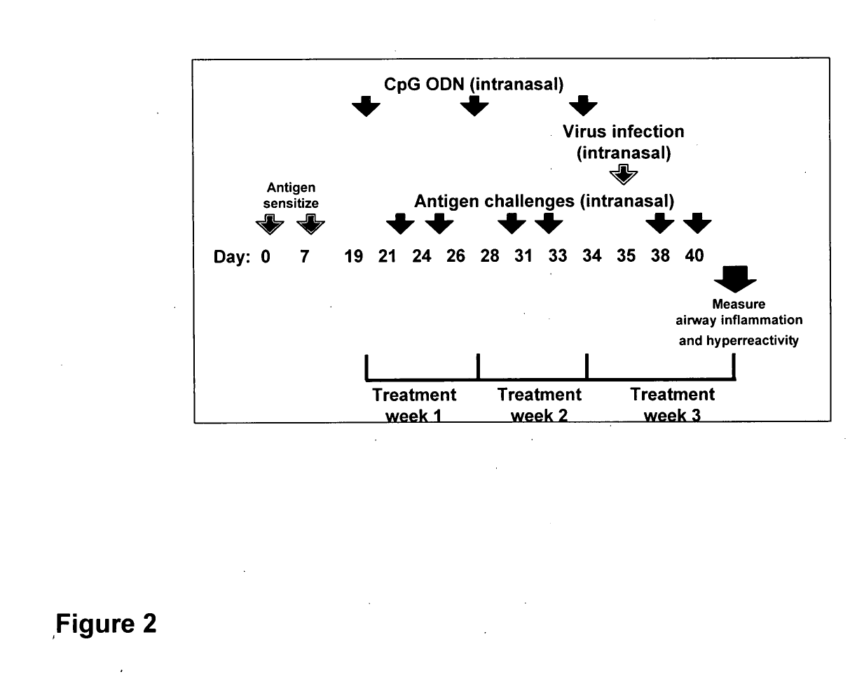

FIG. 3 is a series of graphs depicting IFN-α ((FIG. 3a), IFN-γ (FIG. 3b), and IP-10 (FIG. 3c) induction, and a second series of graphs depicting the upregulation of for 2′5′-oligoadenylate synthetase (FIG. 3d), Mx1 (FIG. 3e), and indoleamine 2,3-dioxygenase (FIG. 3f) in mouse lung. The x-axes represent μg of oligonucleotide per kg of mouse. The y-axes represent cytokine in pg/ml (FIGS. 3a-3c) or the amount of RNA as a ratio of GAPDH RNA (FIGS. 3d-3f).

FIG. 4a is a graph depicting viral nuclear protein titer in mouse lungs. The x-axis represents μg of oligonucleotide per kg of mouse (infected or uninfected) and the y-axis represents absorbance. FIGS. 4b and 4c are graphs showing neutrophils and mononuclear cells, respectively, that are present in bronchoalveolar lavage fluid. The x-axes represent μg of oligonucleotide per kg of mouse (infected or uninfected) and the y-axes represent numbers of cells×10³/ml.

FIG. 5 is a series of graphs depicting total cells accumulated in response to treatment, including total leukocytes (FIG. 5a), neutrophils (FIG. 5b), and mononuclear cells (FIG. 5c) in bronchoaveolar lavage fluid in antigen challenged and virus infected mice. The x-axes represent challenge categories of mice and the y-axes represent numbers of cells×10⁶/ml (5a) or ×10³/ml (5b and 5c).

FIG. 6a is a graph depicting methacholine-induced increase in airway resistance. The x-axis represents mg/ml methacholine and the y-axis represents airway resistance as % of unchallenged control. FIG. 6b shows the baseline airway resistance. FIG. 6c shows areas under the methacholine dose-response curve. Results are presented as mean±SEM (n=7-9).). * P<0.05 compared with group indicated (Mann-Whitney two-tailed test).

FIG. 7 is a series of graphs depicting total cells accumulated in response to treatment, including total leukocytes (FIG. 7a), eosinophils (FIG. 7b), neutrophils (FIG. 7c), and mononuclear cells (FIG. 7d) as well as mouse body weight (FIG. 7e). The x-axes represent challenge categories of mice.

FIG. 8 is a series of graphs demonstrating induction of TLR9-associated cytokines in mouse airways in vivo. FIG. 8a shows IFNα, FIG. 8b shows IFNγ, FIG. 8c shows IP-10, FIG. 8d shows IL-6, and FIG. 8e shows IL-12p40. Results are presented as mean±SEM (n=10). The x-axes represent μg of oligonucleotide per kg of mouse and the y-axes represent cytokine concentration in pg/ml.

FIG. 9 is a series of graphs demonstrating induction of cytokines ex vivo.

FIG. 9a shows IL-5, FIG. 9b shows IL-13, and FIG. 9c shows IFNγ. Results are presented as mean±SEM (n=7-8). * P<0.05 compared with vehicle-treated group (Kruskal-Wallis test followed by Dunn's test for multiple comparisons). The x-axes represent μg of oligonucleotide per kg of mouse and the y-axes represent cytokine concentration in pg/ml.

FIG. 10 is two graphs showing suppression of antigen-induced accumulations of eosinophils and lymphocytes in mouse airways in vivo by SEQ ID NO: 10. FIG. 10a shows IgE production and FIG. 10b shows IgG2a production. Results are presented as mean±SEM (n=9-10). * P<0.05 compared with vehicle-treated group (Kruskal-Wallis test followed by Dunn's test for multiple comparisons). The x-axes represent μg of oligonucleotide per kg of mouse (sensitized or unsensitized) and the y-axes represent absorbance units as a measurement of serum antibody titer.

FIG. 11 is four graphs demonstrating the accumulations of eosinophils and lymphocytes in mouse airways in vivo after administration of SEQ ID NO: 10. FIG. 11a shows total leukocytes present, FIG. 11b shows eosinophils, FIG. 11c shows CD4-positive T cells, and FIG. 11d shows B cells. Results are presented as mean±SEM (n=6). * P<0.05 compared with vehicle-treated group (Kruskal-Wallis test followed by Dunn's test for multiple comparisons). The x-axes represent μg of oligonucleotide per kg of mouse (sensitized or unsensitized) and the y-axes represent number of cells.

### DETAILED DESCRIPTION

Toll-like receptor 9 (TLR9) allows discrete populations of immune cells to recognize unmethylated CpG oligodeoxynucleotides or oligonucleotides (CpG ODN) and to activate host defense mechanisms and initiate immune effects, resulting in suppressed Th2-type responses. Different classes of CpG ODN have been described on the basis of structure and activity characteristics. C-class CpG ODN generally have a 5′ end stimulatory sequence containing at least one CpG motif, and a GC-rich palindrome. C-class CpG ODN induce very high titers of interferon alpha (IFNα) from immune cells.

According to some aspects of the invention, it has been discovered that C-class CpG ODN are of particular value as a novel therapy for upper respiratory tract infections and preferably viral infections as they exacerbate allergic asthma. The data presented in the Examples below, have demonstrated that when dosed into mouse airways, a C-class CpG ODN can induce IFN-associated genes known to have immune-modifying and/or anti-viral activities. In particular, 2′5′-oligoadenylate synthetase and Mx1 (mouse homologue of MxA) are known to have marked anti-viral activity. In our mouse models, a C-class CpG ODN showed protective effects against influenza infection, and suppressed the exacerbated airway inflammation induced by combined antigen challenge and influenza infection.

Thus, in some aspects the invention relates to methods for treating infectious disease exacerbated asthma, and in particular viral exacerbated asthma. Bacterial, viral, and fungal infections exacerbate and/or induce asthma. Infectious disease exacerbated asthma is a condition which occurs in an asthmatic subject. The asthmatic subject, one who has been diagnosed with asthma or is otherwise susceptible to asthma, when exposed to an infectious agent experiences an asthmatic response or an existing/ongoing asthmatic attack is worsened.

Thus, the invention in one aspect involves the finding that CpG immunostimulatory oligonucleotides are useful in treating infectious disease exacerbated asthma.

In some embodiments the subject is at risk of viral infection. A subject at risk of viral infection is one who has any risk of exposure to an infection causing pathogen. For instance, a subject at risk may be a subject who is planning to travel to an area where a particular type of infectious agent is found or it may be a subject who through lifestyle or medical procedures is exposed to bodily fluids which may contain infectious organisms or directly to the organism or even any subject living in an area where an infectious organism or an allergen has been identified. Subjects at risk of developing infection also include general populations to which a medical agency recommends vaccination with a particular infectious organism antigen. A subject at risk of viral infection may be identified in a variety of ways, such as by a medical worker. Medical workers include doctors, nurses, technicians and any other practitioners in the medical field. The subject at risk of a viral infection may also be identified based on exposure to a risk factor for viral infection.

In aspects of the invention the method for identifying a risk factor for viral infection is directed at treating subjects in anticipation of exposure to a viral agent or season (e.g., in anticipation of the flu and cold season). Such seasonal times are generally known and more specifically determined on an annual basis.

A subject having an infection is a subject that has been exposed to an infectious pathogen and has acute or chronic detectable levels of the pathogen in the body. An infectious disease, as used herein, is a disease arising from the presence of a foreign microorganism in the body.

A subject at risk of developing asthma includes those subjects that have been identified as having asthma but that don't have the active disease during the CpG immunostimulatory oligonucleotide treatment as well as subjects that are considered to be at risk of developing these diseases because of genetic or environmental factors.

Th2 cytokines, especially IL-4 and IL-5 are elevated in the airways of asthmatic subjects. These cytokines promote important aspects of the asthmatic inflammatory response, including IgE isotope switching, eosinophil chemotaxis and activation and mast cell growth. Th1 cytokines, especially IFN-γ and IL-12, can suppress the formation of Th2 clones and production of Th2 cytokines. Asthma refers to a disorder of the respiratory system characterized by inflammation, narrowing of the airways and increased reactivity of the airways to inhaled agents. Asthma is frequently, although not exclusively associated with atopic or allergic symptoms.

A subject shall mean a human or vertebrate animal including but not limited to a dog, cat, horse, cow, pig, sheep, goat, turkey, chicken, primate, e.g., monkey, and fish (aquaculture species), e.g. salmon.

As used herein, the term treat, treated, or treating when used with respect to an disorder such as an infectious disease or asthma refers to a prophylactic treatment which increases the resistance of a subject to development of the disease (e.g., to infection with a pathogen) or, in other words, decreases the likelihood that the subject will develop the disease (e.g., become infected with the pathogen) as well as a treatment after the subject has developed the disease in order to fight the disease (e.g., reduce or eliminate the infection) or prevent the disease from becoming worse.

Examples of viruses that have been found in humans include but are not limited to: Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviradae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxyiridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Both gram negative and gram positive bacteria serve as antigens in vertebrate animals. Such gram positive bacteria include, but are not limited to, Pasteurella species, Staphylococci species, and Streptococcus species. Gram negative bacteria include, but are not limited to, Escherichia coli, Pseudomonas species, and Salmonella species. Specific examples of infectious bacteria include but are not limited to, Helicobacter pyloris, Borelia burgdorferi, Legionella pneumophilia, Mycobacteria sps (e.g. M tuberculosis, M avium, M intracellulare, M kansaii, M gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, and Actinomyces israelli.

Examples of fungi include Cryptococcus neoformans, Histoplasma capsulatum, Coccidioides immitis, Blastomyces dermatitidis, Chlamydia trachomatis, Candida albicans.

Other infectious organisms (i.e., protists) include Plasmodium spp. such as Plasmodium falciparum, Plasmodium malariae, Plasmodium ovale, and Plasmodium vivax and Toxoplasma gondii. Blood-borne and/or tissues parasites include Plasmodium spp., Babesia microti, Babesia divergens, Leishmania tropica, Leishmania spp., Leishmania braziliensis, Leishmania donovani, Trypanosoma gambiense and Trypanosoma rhodesiense (African sleeping sickness), Trypanosoma cruzi (Chagas' disease), and Toxoplasma gondii.

Other medically relevant microorganisms have been described extensively in the literature, e.g., see C. G. A Thomas, Medical Microbiology, Bailliere Tindall, Great Britain 1983, the entire contents of which is hereby incorporated by reference.

In some instances the viral exacerbated asthma is caused by a respiratory virus and in particular an upper respiratory virus such as influenza. Optionally the respiratory virus may not be RSV (respiratory syncicial virus).

The method for treating viral exacerbated asthma may also include the use of a combination of CpG oligonucleotides with anti-microbials or a non-CpG asthma therapy such as an asthma medicament. The alternative therapeutic, i.e. the anti-microbial or asthma medicament may be administered at a different time than the CpG oligonucleotide or at the same time as the CpG oligonucleotide.

The asthmatic subject is administered an effective amount of a CpG oligonucleotide for treating viral exacerbated asthma. If a combination of therapeutics is administered the CpG oligonucleotide may be administered to the subject in an amount effective to prevent viral infection and the asthma medicament may be administered to the subject when symptoms of allergy or asthma appear. Thus, the CpG oligonucleotide may be administered to the subject and then the asthma medicament may be subsequently administered to the subject or they are administered together at the same time.

The CpG oligonucleotides contain specific sequences found to elicit an immune response. These specific sequences that elicit an immune response are referred to as “immunostimulatory motifs”, and the oligonucleotides that contain immunostimulatory motifs are referred to as “immunostimulatory oligonucleotide molecules” and, equivalently, “immunostimulatory oligonucleotides”. The immunostimulatory oligonucleotides of the invention thus include at least one immunostimulatory motif. In a preferred embodiment the immunostimulatory motif is an “internal immunostimulatory motif”. The term “internal immunostimulatory motif” refers to the position of the motif sequence within a longer oligonucleotide sequence, which is longer in length than the motif sequence by at least one nucleotide linked to both the 5′ and 3′ ends of the immunostimulatory motif sequence.

The CpG oligonucleotides include at least one unmethylated CpG dinucleotide. An oligonucleotide containing at least one unmethylated CpG dinucleotide is a oligonucleotide molecule which contains an unmethylated cytosine-guanine dinucleotide sequence (i.e., “CpG DNA” or DNA containing a 5′ cytosine followed by 3′ guanine and linked by a phosphate bond) and activates the immune system. The entire CpG oligonucleotide can be unmethylated or portions may be unmethylated but at least the C of the 5′ CG 3′ must be unmethylated.

The methods of the invention may embrace the use of A class, B class and C class CpG immunostimulatory oligonucleotides. As to CpG oligonucleotides, it has recently been described that there are different classes of CpG oligonucleotides. One class is potent for activating B cells but is relatively weak in inducing IFN-α and NK cell activation; this class has been termed the B class. The B class CpG oligonucleotides typically are fully stabilized and include an unmethylated CpG dinucleotide within certain preferred base contexts. See, e.g., U.S. Pat. Nos. 6,194,388; 6,207,646; 6,214,806; 6,218,371; 6,239,116; and 6,339,068. Another class is potent for inducing IFN-α and NK cell activation but is relatively weak at stimulating B cells; this class has been termed the A class. The A class CpG oligonucleotides typically have stabilized poly-G sequences at 5′ and 3′ ends and a palindromic phosphodiester CpG dinucleotide-containing sequence of at least 6 nucleotides. See, for example, published patent application PCT/JUS00/26527 (WO 01/22990). Yet another class of CpG oligonucleotides activates B cells and NK cells and induces IFN-α; this class has been termed the C-class. The C-class CpG oligonucleotides, as first characterized, typically are fully stabilized, include a B class-type sequence and a GC-rich palindrome or near-palindrome. This class has been described in U.S. patent application Ser. No. 10/224,523 filed on Aug. 19, 2002, and U.S. Ser. No. 10/978,282 filed Oct. 29, 2004 the entire contents of which are incorporated herein by reference.

“A class” CpG immunostimulatory oligonucleotides have been described in U.S. Non-Provisional patent application Ser. No. 09/672,126 and published PCT application PCT/US00/26527 (WO 01/22990), both filed on Sep. 27, 2000 as well as in U.S. Pat. No. 6,207,646 B 1. These oligonucleotides are characterized by the ability to induce high levels of interferon-alpha while having minimal effects on B cell activation. The A class CpG immunostimulatory oligonucleotides do not necessarily contain a hexamer palindrome GACGTC, AGCGCT, or AACGTT described by Yamamoto and colleagues. Yamamoto S et al. J. Immunol 148:4072-6 (1992).

B class CpG immunostimulatory oligonucleotides strongly activate human B cells but have minimal effects inducing interferon-α. B class CpG immunostimulatory oligonucleotides have been described in USPs 6,194,388 B1 and 6,239,116 B1, issued on Feb. 27, 2001 and May 29, 2001 respectively.

In one embodiment the invention provides a B class CpG oligonucleotide represented by at least the formula:
5′ X₁X₂CGX₃X₄ 3′
wherein X₁, X₂, X₃, and X₄ are nucleotides. In one embodiment X₂ is adenine, guanine, or thymine. In another embodiment X₃ is cytosine, adenine, or thymine.

In another embodiment the invention provides an isolated B class CpG oligonucleotide represented by at least the formula:
5t N₁X₁X₂CGX₃X₄N₂ 3′
wherein X₁, X₂, X₃, and X₄ are nucleotides and N is any nucleotide and N₁ and N₂ are oligonucleotide sequences composed of from about 0-25 N's each. In one embodiment X₁X₂ is a dinucleotide selected from the group consisting of: GpT, GpG, GpA, ApA, ApT, ApG, CpT, CpA, CpG, TpA, TpT, and TpG; and X₃X₄ is a dinucleotide selected from the group consisting of: TpT, ApT, TpG, ApG, CpG, TpC, ApC, CpC, TpA, ApA, and CpA. Preferably X₁X₂ is GpA or GpT and X₃X₄ is TpT. In other embodiments X₁ or X₂ or both are purines and X₃ or X₄ or both are pyrimidines or X₁X₂ is GpA and X₃ or X₄ or both are pyrimidines. In another preferred embodiment X₁X₂ is a dinucleotide selected from the group consisting of: TpA, ApA, ApC, ApG, and GpG. In yet another embodiment X₃X₄ is a dinucleotide selected from the group consisting of: TpT, TpA, TpG, ApA, ApG, GpA, and CpA. X₁X₂ in another embodiment is a dinucleotide selected from the group consisting of: TpT, TpG, ApT, GpC, CpC, CpT, TpC, GpT and CpG; X₃ is a nucleotide selected from the group consisting of A and T and X₄ is a nucleotide, but wherein when X₁X₂ is TpC, GpT, or CpG, X₃X₄ is not TpC, ApT or ApC. In some embodiments the oligonucleotide has a 5′TC.

In another preferred embodiment the CpG oligonucleotide has the sequence 5′ TCN₁TX₁X₂CGX₃X₄ 3′ (SEQ ID NO 56). The CpG oligonucleotides of the invention in some embodiments include X₁X₂ selected from the group consisting of GpT, GpG, GpA and ApA and X₃X₄ is selected from the group consisting of TpT, CpT and TpC.

The B class CpG oligonucleotide sequences of the invention are those broadly described above as well as disclosed in PCT Published Patent Applications PCT/US95/01570 and PCT/US97/19791, and U.S. Pat. No. 6,194,388 B1 and U.S. Pat. No. 6,239,116 B1, issued Feb. 27, 2001 and May 29, 2001 respectively. Exemplary sequences include but are not limited to those disclosed in these latter applications and patents.

The C class immunostimulatory oligonucleotides contain at least two distinct motifs and have unique and desirable stimulatory effects on cells of the immune system. Some of these ODN have both a traditional “stimulatory” CpG sequence and a “GC-rich” or “B-cell neutralizing” motif. These combination motif oligonucleotides have immune stimulating effects that fall somewhere between those effects associated with traditional “class B” CpG ODN, which are strong inducers of B cell activation and dendritic cell (DC) activation, and those effects associated with class A CpG ODN which are strong inducers of IFN-α and natural killer (NK) cell activation but relatively poor inducers of B-cell and DC activation. While preferred class B CpG ODN often have phosphorothioate backbones and preferred class A CpG ODN have mixed or chimeric backbones, the C class of combination motif immune stimulatory oligonucleotides may have either stabilized, e.g., phosphorothioate, chimeric, or phosphodiester backbones, and in some preferred embodiments, they have semi-soft backbones.

The stimulatory domain or motif may be defined by a formula: 5′ X₁DCGHX₂ 3′. D is a nucleotide other than C. C is cytosine. G is guanine. H is a nucleotide other than G.

X₁ and X₂ are any oligonucleotide sequence 0 to 10 nucleotides long. X₁ may include a CG, in which case there is preferably a T immediately preceding this CG. In some embodiments DCG is TCG. X₁ is preferably from 0 to 6 nucleotides in length. In some embodiments X₂ does not contain any poly G or poly A motifs. In other embodiments the immunostimulatory oligonucleotide has a poly-T sequence at the 5′ end or at the 3′ end. As used herein, “poly-A” or “poly-T” shall refer to a stretch of four or more consecutive A's or T's respectively, e.g., 5′ AAAA 3′ or 5′ TTTT 3′. As used herein, “poly-G end” shall refer to a stretch of four or more consecutive G's, e.g., 5′ GGGG 3′, occurring at the 5′ end or the 3′ end of a oligonucleotide. As used herein, “poly-G oligonucleotide” shall refer to a oligonucleotide having the formula 5′ X₁X₂GGGX₃X₄ 3′ wherein X₁, X₂, X₃, and X₄ are nucleotides and preferably at least one of X₃ and X₄ is a G.

Some preferred designs for the B cell stimulatory domain under this formula comprise TTTTTCG, TCG, TTCG, TTTCG, TTTTCG, TCGT, TTCGT, TTTCGT, TCGTCGT.

The second motif of the oligonucleotide is referred to as either P or N and is positioned immediately 5′ to X₁ or immediately 3′ to X₂.

N is a B-cell neutralizing sequence that begins with a CGG trinucleotide and is at least 10 nucleotides long. A B-cell neutralizing motif includes at least one CpG sequence in which the CG is preceded by a C or followed by a G (Krieg A M et al. (1998) Proc Natl Acad Sci USA 95:12631-12636) or is a CG containing DNA sequence in which the C of the CG is methylated. As used herein, “CpG” shall refer to a 5′ cytosine (C) followed by a 3′ guanine (G) and linked by a phosphate bond. At least the C of the 5′ CG 3′ must be unmethylated. Neutralizing motifs are motifs which have some degree of immunostimulatory capability when present in an otherwise non-stimulatory motif, but, which when present in the context of other immunostimulatory motifs serve to reduce the immunostimulatory potential of the other motifs.

P is a GC-rich palindrome containing sequence at least 10 nucleotides long. As used herein, “palindrome” and, equivalently, “palindromic sequence” shall refer to an inverted repeat, i.e., a sequence such as ABCDEE′D′C′B′A′ in which A and A′, B and B′, etc., are bases capable of forming the usual Watson-Crick base pairs.

As used herein, “GC-rich palindrome” shall refer to a palindrome having a base composition of at least two-thirds G's and C's. In some embodiments the GC-rich domain is preferably 3′ to the “B cell stimulatory domain”. In the case of a 10-base long GC-rich palindrome, the palindrome thus contains at least 8 G's and C's. In the case of a 12-base long GC-rich palindrome, the palindrome also contains at least 8 G's and C's. In the case of a 14-mer GC-rich palindrome, at least ten bases of the palindrome are G's and C's. In some embodiments the GC-rich palindrome is made up exclusively of G's and C's.

In some embodiments the GC-rich palindrome has a base composition of at least 81 percent G's and C's. In the case of such a 10-base long GC-rich palindrome, the palindrome thus is made exclusively of G's and C's. In the case of such a 12-base long GC-rich palindrome, it is preferred that at least ten bases (83 percent) of the palindrome are G's and C's. In some preferred embodiments, a 12-base long GC-rich palindrome is made exclusively of G's and C's. In the case of a 14-mer GC-rich palindrome, at least twelve bases (86 percent) of the palindrome are G's and C's. In some preferred embodiments, a 14-base long GC-rich palindrome is made exclusively of G's and C's. The C's of a GC-rich palindrome can be unmethylated or they can be methylated.

In general this domain has at least 3 Cs and Gs, more preferably 4 of each, and most preferably 5 or more of each. The number of Cs and Gs in this domain need not be identical. It is preferred that the Cs and Gs are arranged so that they are able to form a self-complementary duplex, or palindrome, such as CCGCGCGG. This may be interrupted by As or Ts, but it is preferred that the self-complementarity is at least partially preserved as for example in the motifs CGACGTTCGTCG (SEQ ID NO: 49) or CGGCGCCGTGCCG (SEQ ID NO: 50). When complementarity is not preserved, it is preferred that the non-complementary base pairs be TG. In a preferred embodiment there are no more than 3 consecutive bases that are not part of the palindrome, preferably no more than 2, and most preferably only 1. In some embodiments the GC-rich palindrome includes at least one CGG trimer, at least one CCG trimer, or at least one CGCG tetramer. In other embodiments the GC-rich palindrome is not CCCCCCGGGGGG (SEQ ID NO: 51) or GGGGGGCCCCCC (SEQ ID NO: 52), CCCCCGGGGG (SEQ ID NO: 53) or GGGGGCCCCC (SEQ ID NO: 54).

At least one of the G's of the GC rich region may be substituted with an inosine (I). In some embodiments P includes more than one I.

In certain embodiments the immunostimulatory oligonucleotide has one of the following formulas 5′ NX₁DCGHX₂ 3′, 5′ X₁DCGHX₂N 3′, 5′ PX₁DCGHX₂ 3′, 5′ X₁DCGHX₂P 3′, 5′ X₁DCGHX₂PX₃ 3′, 5′ X₁DCGHPX₃ 3′, 5′ DCGHX₂PX₃ 3′, 5′ TCGHX₂PX₃ 3′, 5′ DCGHPX₃ 3′, or 5′ DCGHP 3′.

In other aspects the invention provides immune stimulatory oligonucleotides which are defined by a formula: 5′ N₁PyGN₂P 3′. N₁ is any sequence 1 to 6 nucleotides long. Py is a pyrimidine. G is guanine. N₂ is any sequence 0 to 30 nucleotides long. P is a GC-rich palindrome containing sequence at least 10 nucleotides long.

N₁ and N₂ may contain more than 50% pyrimidines, and more preferably more than 50% T. N₁ may include a CG, in which case there is preferably a T immediately preceding this CG. In some embodiments N₁PyG is TCG (such as ODN 5376, which has a 5′ TCGG), and most preferably a TCGN₂, where N₂ is not G.

N₁PyGN₂P may include one or more inosine (I) nucleotides. Either the C or the G in N1 may be replaced by inosine, but the CpI is preferred to the IpG. For inosine substitutions such as IpG, the optimal activity may be achieved with the use of a “semi-soft” or chimeric backbone, where the linkage between the IG or the CI is phosphodiester. N₁ may include at least one CI, TCI, IG or TIG motif.

In certain embodiments N₁PyGN₂ is a sequence selected from the group consisting of TTTTTCG, TCG, TTCG, TTTCG, TTTTCG, TCGT, TTCGT, TTTCGT, and TCGTCGT.

C-Class ODN are also described in U.S. patent application Ser. No. 10/978,283 filed on Oct. 28, 2004. The nucleic acids described therein are all incorporated by reference.

Some non limiting examples of CpG oligonucleotides useful according to the invention include:

| | |
|---|---|
| SEQ ID | |
| NO | Sequence |

| | |
|---|---|
| 1 | T*C_G*C_G*T*C_G*T*T*C_G*G*C*G*C_G*C*G*C*C*G |
| 2 | T*C_G*T*C_G*A*C_G*T*T*C_G*G*C*G*C_G*C*G*C*C*G |
| 3 | T*C_G*G*A*C_G*T*T*C_G*G*C*G*C_G*C*G*C*C*G |
| 4 | T*C_G*G*A*C_G*T*T*C_G*G*C*G*C*G*C*C*G |
| 5 | T*C_G*C_G*T*C_G*T*T*C_G*G*C*G*C*G*C*C*G |
| 6 | T*C_G*A*C_G*T*T*C_G*G*C*G*C_G*C*G*C*C*G |
| 7 | T*C_G*A*C G*T*T*C_G*G*C*G*C*G*C*C*G |
| 8 | T*C_G*C_G*T*C_G*T*T*C_G*G*C*G*C*C*G |
| 9 | T*C_G*C_G*A*C_G*T*T*C_G*G*C*G*C_G*C*G*C*C*G |
| 10 | T*C_G*T*C_G*T*C_G*T*T*C_G*G*C*G*C_G*C*G*C*C*G |
| 11 | T*C*G*T*C*G*T*T*T*T*G*A*C*G*T*T*T*T*G*T*C*G*T*T |
| 12 | T*C*G*T*C*G*T*T*T*T*G*T*C*G*T*T*T*T*G*T*C*G*T*T |
| 13 | T*C*G*T*C_G*T*T*T*T*A*C_G*G*C*G*C*C_G*T*G*C*C*G |
| 14 | T*C_G*T*C_G*T*T*T*T*G*A*C_G*T*T*T*T*G*T*C_G*T*T |
| 15 | T*C_G*T*C_G*A*C_G*T*T*C_G*G*C*G*C_G*C*G*C*C*G |
| 16 | T*C_G_A_C_G_T_C_G_T_G_G*G*G*G |
| 17 | T*C*G*A*C*G*T*C*G*A*C*G*T*G*A*C*G*T*G |
| 18 | T*C*G*A*C*G*T*C*G*A*C*G*T*G*A*C*G |
| 19 | T*T*C_G*T*C_G*T*T*T*T_G*T*C_G*T*T |
| 20 | T*T*T*C_G*T*C_G*T*T*T*C_G*T*C_G*T*T |
| 21 | T*C*G*T*C_G*T*A*C_G*G*C*G*C*C_G*T*G*C*C*G |
| 22 | T*C*G*T*C_G*T*T*A*C_G*G*C*G*C*C_G*T*G*C*C*G |
| 23 | T*C*G*A*C*G*T*C*G*A*C*G*T*G*A*C*G*T*T |
| 24 | T*C*G*T*C_G*A*C_G*A*T*C_G*G*C*G*C*C_G*T*G*C*C*G |
| 25 | T*C*G*T*C*G*A*C*G*A_T_C*G*G*C*G*C*C*G*T*G*C*C*G |
| 26 | T*C*G*A*C_G*T*C*G*A*C_G*T*G*A*C*G*T*T |
| 27 | T*C*G*A*C_G*T*C*G*A*C*G*T_G*A*C*G*T*T |
| 28 | T*C*G*T*C_G*T*T*T*A*C_G*G*C*G*C*C_G*T*G*C*C*G*T |
| 29 | T*C_G*T*C_G*T*T*T*T*G*A*C_G*T*T*T*T*G*T*C_G*T*T |
| 30 | T*C_G*T*T*T*T*G*A*C_G*T*T*T*T*G*T*C_G*T*T |
| 31 | T*C_G*T*T*T*T*G*A*C_G*T*T |
| 32 | T*C_G*T*C_G*T*T*T_T*G*A*C_G*T*T*T*T*G*T*C_G*T*T |
| 33 | T*C_G*T*C_G*T*T*T_T*G*A*C_G*T*T*T_T*G*T*C_G*T*T |
| 34 | T*C_G*T*C_G*T_T*T_T*G_A*C_G*T_T*T_T*G_T*C_G*T*T |
| 35 | T_C_G_T_C_G_T_T_T_T_G_A_C_G_T_T_T_T_G_T_C_G_T_T |
| 36 | G*T*C_G*T*T*T*T*G*A*C_G*T*T*T*T*G*T*C_G*T*T |
| 37 | T*C_G*T*C_G*T*T*T*T*G*A*C_G*T*T*T*T*G*T*C |
| 38 | T*C_G*T*C_G*T*T*T*T*G*A*C |
| 39 | G*T*T*T*T*G*A*C_G*T*T*T*T*G*T*C |
| 40 | G*T*T*T*T*G*A*C_G*T*T*T*T*G*T*C_G*T*T |
| 41 | G*T*C_G*T*T*T*T*G*A*C_G*T*T |
| 42 | T*C_G*T*T*T*T*G*A*C_G*T*T*T*T*G*T*C |
| 43 | G*T*C_G*T*T*T*T*G*A*C_G*T*T*T*T*G*T*C |
| 44 | G*T*C_G*T*T*T*T*G*A*C |
| 45 | C_G*T*C_G*T*T*T*T*G*A*C_G*T*T*T*T*G*T*C_G*T*T |
| 46 | T*C*G*A*T*C*G*T*T*T*T_T_C_G*T*G*C*G*T*T*T*T*T |
| 47 | T*C*G*C*G*A*C_G*T*T*C*G*C*G*C_G*C*G*C*G |
| 48 | T*C_G*G*A*C_G*T*T*C_G*G*C*G*C*G*C*C*G |
| 49 | CGACGTTCGTCG |
| 50 | CGGCGCCGTGCCG |
| 51 | CCCCCCGGGGGG |
| 52 | GGGGGGCCCCCC |
| 53 | CCCCCGGGGG |
| 54 | GGGGGCCCCC |
| 55 | T*G_C*T*C_G*T*C_G*T*T*C_G*G*C*G*C_G*C*G*C*C*G |
| 56 | TCXTXXCGXX |
| 57 | T*C_G*T*C_G*T*T*C_G*G*C*G*C*G*C*C*G |

The immunostimulatory oligonucleotide molecules may have a chimeric backbone. For purposes of the instant invention, a chimeric backbone refers to a partially stabilized backbone, wherein at least one internucleotide linkage is phosphodiester or phosphodiester-like, and wherein at least one other internucleotide linkage is a stabilized internucleotide linkage, wherein the at least one phosphodiester or phosphodiester-like linkage and the at least one stabilized linkage are different. Since boranophosphonate linkages have been reported to be stabilized relative to phosphodiester linkages, for purposes of the chimeric nature of the backbone, boranophosphonate linkages can be classified either as phosphodiester-like or as stabilized, depending on the context. For example, a chimeric backbone according to the instant invention could in one embodiment include at least one phosphodiester (phosphodiester or phosphodiester-like) linkage and at least one boranophosphonate (stabilized) linkage. In another embodiment a chimeric backbone according to the instant invention could include boranophosphonate (phosphodiester or phosphodiester-like) and phosphorothioate (stabilized) linkages. A “stabilized internucleotide linkage” shall mean an internucleotide linkage that is relatively resistant to in vivo degradation (e.g., via an exo- or endo-nuclease), compared to a phosphodiester internucleotide linkage. Preferred stabilized internucleotide linkages include, without limitation, phosphorothioate, phosphorodithioate, methylphosphonate, and methylphosphorothioate. Other stabilized internucleotide linkages include, without limitation: peptide, alkyl, dephospho, and others as described above.

Modified backbones such as phosphorothioates may be synthesized using automated techniques employing either phosphoramidate or H-phosphonate chemistries. Aryl- and alkyl-phosphonates can be made, e.g., as described in U.S. Pat. No. 4,469,863; and alkylphosphotriesters (in which the charged oxygen moiety is alkylated as described in U.S. Pat. No. 5,023,243 and European Patent No. 092,574) can be prepared by automated solid phase synthesis using commercially available reagents. Methods for making other DNA backbone modifications and substitutions have been described. Uhlmann E et al. (1990) Chem Rev 90:544; Goodchild J (1990) Bioconjugate Chem 1:165. Methods for preparing chimeric oligonucleotides are also known. For instance patents issued to Uhlmann et al have described such techniques.

Mixed backbone modified ODN may be synthesized using a commercially available DNA synthesizer and standard phosphoramidite chemistry. (F. E. Eckstein, “Oligonucleotides and Analogues—A Practical Approach” IRL Press, Oxford, UK, 1991, and M. D. Matteucci and M. H. Caruthers, Tetrahedron Lett. 21, 719 (1980)) After coupling, PS linkages are introduced by sulfurization using the Beaucage reagent (R. P. Iyer, W. Egan, J. B. Regan and S. L. Beaucage, J. Am. Chem. Soc. 112, 1253 (1990)) (0.075 M in acetonitrile) or phenyl acetyl disulfide (PADS) followed by capping with acetic anhydride, 2,6-lutidine in tetrahydrofurane (1:1:8; v:v:v) and N-methylimidazole (16% in tetrahydrofurane). This capping step is performed after the sulfurization reaction to minimize formation of undesired phosphodiester (PO) linkages at positions where a phosphorothioate linkage should be located. In the case of the introduction of a phosphodiester linkage, e.g. at a CpG dinucleotide, the intermediate phosphorous-III is oxidized by treatment with a solution of iodine in water/pyridine. After cleavage from the solid support and final deprotection by treatment with concentrated ammonia (15 hrs at 50° C.), the ODN are analyzed by HPLC on a Gen-Pak Fax column (Millipore-Waters) using a NaCl-gradient (e.g. buffer A: 10 mM NaH₂PO₄ in acetonitrile/water=1:4/v:v pH 6.8; buffer B: 10 mM NaH₂PO₄, 1.5 M NaCl in acetonitrile/water=1:4/v:v; 5 to 60% B in 30 minutes at 1 ml/min) or by capillary gel electrophoresis. The ODN can be purified by HPLC or by FPLC on a Source High Performance column (Amersham Pharmacia). HPLC-homogeneous fractions are combined and desalted via a C18 column or by ultrafiltration. The ODN was analyzed by MALDI-TOF mass spectrometry to confirm the calculated mass.

The oligonucleotides of the invention can also include other modifications. These include nonionic DNA analogs, such as alkyl- and aryl-phosphates (in which the charged phosphonate oxygen is replaced by an alkyl or aryl group), phosphodiester and alkylphosphotriesters, in which the charged oxygen moiety is alkylated. Oligonucleotides which contain diol, such as tetraethyleneglycol or hexaethyleneglycol, at either or both termini have also been shown to be substantially resistant to nuclease degradation.

In some embodiments the oligonucleotides may be soft or semi-soft oligonucleotides. A soft oligonucleotide is an immunostimulatory oligonucleotide having a partially stabilized backbone, in which phosphodiester or phosphodiester-like internucleotide linkages occur only within and immediately adjacent to at least one internal pyrimidine-purine dinucleotide (YZ). Preferably YZ is YG, a pyrimidine-guanosine (YG) dinucleotide. The at least one internal YZ dinucleotide itself has a phosphodiester or phosphodiester-like internucleotide linkage. A phosphodiester or phosphodiester-like internucleotide linkage occurring immediately adjacent to the at least one internal YZ dinucleotide can be 5′, 3′, or both 5′ and 3′ to the at least one internal YZ dinucleotide.

In particular, phosphodiester or phosphodiester-like internucleotide linkages involve “internal dinucleotides”. An internal dinucleotide in general shall mean any pair of adjacent nucleotides connected by an internucleotide linkage, in which neither nucleotide in the pair of nucleotides is a terminal nucleotide, i.e., neither nucleotide in the pair of nucleotides is a nucleotide defining the 5′ or 3′ end of the oligonucleotide. Thus a linear oligonucleotide that is n nucleotides long has a total of n−1 dinucleotides and only n−3 internal dinucleotides. Each internucleotide linkage in an internal dinucleotide is an internal internucleotide linkage. Thus a linear oligonucleotide that is n nucleotides long has a total of n−1 internucleotide linkages and only n−3 internal internucleotide linkages. The strategically placed phosphodiester or phosphodiester-like internucleotide linkages, therefore, refer to phosphodiester or phosphodiester-like internucleotide linkages positioned between any pair of nucleotides in the oligonucleotide sequence. In some embodiments the phosphodiester or phosphodiester-like internucleotide linkages are not positioned between either pair of nucleotides closest to the 5′ or 3′ end.

Preferably a phosphodiester or phosphodiester-like internucleotide linkage occurring immediately adjacent to the at least one internal YZ dinucleotide is itself an internal internucleotide linkage. Thus for a sequence N₁ YZ N₂, wherein N₁ and N₂ are each, independent of the other, any single nucleotide, the YZ dinucleotide has a phosphodiester or phosphodiester-like internucleotide linkage, and in addition (a) N₁ and Y are linked by a phosphodiester or phosphodiester-like internucleotide linkage when N₁ is an internal nucleotide, (b) Z and N₂ are linked by a phosphodiester or phosphodiester-like internucleotide linkage when N₂ is an internal nucleotide, or (c) N₁ and Y are linked by a phosphodiester or phosphodiester-like internucleotide linkage when N₁ is an internal nucleotide and Z and N₂ are linked by a phosphodiester or phosphodiester-like internucleotide linkage when N₂ is an internal nucleotide.

Soft oligonucleotides according to the instant invention are believed to be relatively susceptible to nuclease cleavage compared to completely stabilized oligonucleotides. Without meaning to be bound to a particular theory or mechanism, it is believed that soft oligonucleotides of the invention are cleavable to fragments with reduced or no immunostimulatory activity relative to full-length soft oligonucleotides. Incorporation of at least one nuclease-sensitive internucleotide linkage, particularly near the middle of the oligonucleotide, is believed to provide an “off switch” which alters the pharmacokinetics of the oligonucleotide so as to reduce the duration of maximal immunostimulatory activity of the oligonucleotide. This can be of particular value in tissues and in clinical applications in which it is desirable to avoid injury related to chronic local inflammation or immunostimulation, e.g., the kidney.

A semi-soft oligonucleotide is an immunostimulatory oligonucleotide having a partially stabilized backbone, in which phosphodiester or phosphodiester-like internucleotide linkages occur only within at least one internal pyrimidine-purine (YZ) dinucleotide. Semi-soft oligonucleotides generally possess increased immunostimulatory potency relative to corresponding fully stabilized immunostimulatory oligonucleotides. Due to the greater potency of semi-soft oligonucleotides, semi-soft oligonucleotides may be used, in some instances, at lower effective concentations and have lower effective doses than conventional fully stabilized immunostimulatory oligonucleotides in order to achieve a desired biological effect.

It is believed that the foregoing properties of semi-soft oligonucleotides generally increase with increasing “dose” of phosphodiester or phosphodiester-like internucleotide linkages involving internal YZ dinucleotides. Thus it is believed, for example, that generally for a given oligonucleotide sequence with five internal YZ dinucleotides, an oligonucleotide with five internal phosphodiester or phosphodiester-like YZ internucleotide linkages is more immunostimulatory than an oligonucleotide with four internal phosphodiester or phosphodiester-like YG internucleotide linkages, which in turn is more immunostimulatory than an oligonucleotide with three internal phosphodiester or phosphodiester-like YZ internucleotide linkages, which in turn is more immunostimulatory than an oligonucleotide with two internal phosphodiester or phosphodiester-like YZ internucleotide linkages, which in turn is more immunostimulatory than an oligonucleotide with one internal phosphodiester or phosphodiester-like YZ internucleotide linkage. Importantly, inclusion of even one internal phosphodiester or phosphodiester-like YZ internucleotide linkage is believed to be advantageous over no internal phosphodiester or phosphodiester-like YZ internucleotide linkage. In addition to the number of phosphodiester or phosphodiester-like internucleotide linkages, the position along the length of the oligonucleotide can also affect potency. The soft and semi-soft oligonucleotides will generally include, in addition to the phosphodiester or phosphodiester-like internucleotide linkages at preferred internal positions, 5′ and 3′ ends that are resistant to degradation. Such degradation-resistant ends can involve any suitable modification that results in an increased resistance against exonuclease digestion over corresponding unmodified ends. For instance, the 5′ and 3′ ends can be stabilized by the inclusion there of at least one phosphate modification of the backbone. In a preferred embodiment, the at least one phosphate modification of the backbone at each end is independently a phosphorothioate, phosphorodithioate, methylphosphonate, or methylphosphorothioate internucleotide linkage. In another embodiment, the degradation-resistant end includes one or more nucleotide units connected by peptide or amide linkages at the 3′ end.

A phosphodiester internucleotide linkage is the type of linkage characteristic of oligonucleotides found in nature. The phosphodiester internucleotide linkage includes a phosphorus atom flanked by two bridging oxygen atoms and bound also by two additional oxygen atoms, one charged and the other uncharged. Phosphodiester internucleotide linkage is particularly preferred when it is important to reduce the tissue half-life of the oligonucleotide.

A phosphodiester-like internucleotide linkage is a phosphorus-containing bridging group that is chemically and/or diastereomerically similar to phosphodiester. Measures of similarity to phosphodiester include susceptibility to nuclease digestion and ability to activate RNAse H. Thus for example phosphodiester, but not phosphorothioate, oligonucleotides are susceptible to nuclease digestion, while both phosphodiester and phosphorothioate oligonucleotides activate RNAse H. In a preferred embodiment the phosphodiester-like internucleotide linkage is boranophosphate (or equivalently, boranophosphonate) linkage. U.S. Pat. No. 5,177,198; U.S. Pat. No. 5,859,231; U.S. Pat. No. 6,160,109; U.S. Pat. No. 6,207,819; Sergueev et al., (1998) J Am Chem Soc 120:9417-27. In another preferred embodiment the phosphodiester-like internucleotide linkage is diasteromerically pure Rp phosphorothioate. It is believed that diasteromerically pure Rp phosphorothioate is more susceptible to nuclease digestion and is better at activating RNAse H than mixed or diastereomerically pure Sp phosphorothioate. Stereoisomers of CpG oligonucleotides are the subject of co-pending U.S. patent application Ser. No. 09/361,575 filed Jul. 27, 1999, and published PCT application PCT/US99/17100 (WO 00/06588). It is to be noted that for purposes of the instant invention, the term “phosphodiester-like internucleotide linkage” specifically excludes phosphorodithioate and methylphosphonate internucleotide linkages.

As described above the soft and semi-soft oligonucleotides of the invention may have phosphodiester like linkages between C and G. One example of a phosphodiester-like linkage is a phosphorothioate linkage in an Rp conformation. Oligonucleotide p-chirality can have apparently opposite effects on the immune activity of a CpG oligonucleotide, depending upon the time point at which activity is measured. At an early time point of 40 minutes, the Rₚ but not the S_{P} stereoisomer of phosphorothioate CpG oligonucleotide induces JNK phosphorylation in mouse spleen cells. In contrast, when assayed at a late time point of 44 hr, the S_{P} but not the Rₚ stereoisomer is active in stimulating spleen cell proliferation. This difference in the kinetics and bioactivity of the Rₚ and Sₚ stereoisomers does not result from any difference in cell uptake, but rather most likely is due to two opposing biologic roles of the p-chirality. First, the enhanced activity of the Rp stereoisomer compared to the Sp for stimulating immune cells at early time points indicates that the Rₚ may be more effective at interacting with the CpG receptor, TLR9, or inducing the downstream signaling pathways. On the other hand, the faster degradation of the Rp PS-oligonucleotides compared to the Sp results in a much shorter duration of signaling, so that the Sp PS-oligonucleotides appear to be more biologically active when tested at later time points.

A surprisingly strong effect is achieved by the p-chirality at the CpG dinucleotide itself. In comparison to a stereo-random CpG oligonucleotide the congener in which the single CpG dinucleotide was linked in Rp was slightly more active, while the congener containing an Sp linkage was nearly inactive for inducing spleen cell proliferation.

The size (i.e., the number of nucleotide residues along the length of the oligonucleotide) of the immunostimulatory oligonucleotide may also contribute to the stimulatory activity of the oligonucleotide. For facilitating uptake into cells immunostimulatory oligonucleotides preferably have a minimum length of 6 nucleotide residues. Oligonucleotides of any size greater than 6 nucleotides (even many kb long) are capable of inducing an immune response according to the invention if sufficient immunostimulatory motifs are present, since larger oligonucleotides are degraded inside of cells. It is believed by the instant inventors that semi-soft oligonucleotides as short as 4 nucleotides can also be immunostimulatory if they can be delivered to the interior of the cell. In certain preferred embodiments according to the instant invention, the immunostimulatory oligonucleotides are between 4 and 100 nucleotides long. In typical embodiments the immunostimulatory oligonucleotides are between 6 and 40 nucleotides long. In certain embodiments according to the instant invention, the immunostimulatory oligonucleotides are between 6 and 19 nucleotides long. The immunostimulatory oligonucleotides generally have a length in the range of between 4 and 100 and in some embodiments 8 and 40. The length may be in the range of between 16 and 24 nucleotides.

The term “oligonucleotide” also encompasses oligonucleotides with substitutions or modifications, such as in the bases and/or sugars. For example, they include oligonucleotides having backbone sugars that are covalently attached to low molecular weight organic groups other than a hydroxyl group at the 2′ position and other than a phosphate group or hydroxy group at the 5′ position. Thus modified oligonucleotides may include a 2′-O-alkylated ribose group. In addition, modified oligonucleotides may include sugars such as arabinose or 2′-fluoroarabinose instead of ribose. Thus the oligonucleotides may be heterogeneous in backbone composition thereby containing any possible combination of polymer units linked together such as peptide-nucleic acids (which have an amino acid backbone with oligonucleotide bases).

Oligonucleotides also include substituted purines and pyrimidines such as C-5 propyne pyrimidine and 7-deaza-7-substituted purine modified bases. Wagner R W et al. (1996) Nat Biotechnol 14:840-4. Purines and pyrimidines include but are not limited to adenine, cytosine, guanine, thymine, 5-methylcytosine, 5-hydroxycytosine, 5-fluorocytosine, 2-aminopurine, 2-amino-6-chloropurine, 2,6-diaminopurine, hypoxanthine, and other naturally and non-naturally occurring nucleobases, substituted and unsubstituted aromatic moieties. Other such modifications are well known to those of skill in the art.

The immunostimulatory oligonucleotides of the instant invention can encompass various chemical modifications and substitutions, in comparison to natural RNA and DNA, involving a phosphodiester internucleotide bridge, a β-D-ribose unit and/or a natural nucleotide base (adenine, guanine, cytosine, thymine, uracil). Examples of chemical modifications are known to the skilled person and are described, for example, in Uhlmann E et al. (1990) Chem Rev 90:543; “Protocols for Oligonucleotides and Analogs” Synthesis and Properties & Synthesis and Analytical Techniques, S. Agrawal, Ed, Humana Press, Totowa, USA 1993; Crooke ST et al. (1996) Annu Rev Pharmacol Toxicol 36:107-129; and Hunziker J et al. (1995) Mod Synth Methods 7:331-417. An oligonucleotide according to the invention may have one or more modifications, wherein each modification is located at a particular phosphodiester internucleotide bridge and/or at a particular β-D-ribose unit and/or at a particular natural nucleotide base position in comparison to an oligonucleotide of the same sequence which is composed of natural DNA or RNA.

For example, the invention relates to an oligonucleotide which may comprise one or more modifications and wherein each modification is independently selected from:
a) the replacement of a phosphodiester internucleotide bridge located at the 3′ and/or the 5′ end of a nucleotide by a modified internucleotide bridge,
b) the replacement of phosphodiester bridge located at the 3′ and/or the 5′ end of a nucleotide by a dephospho bridge,
c) the replacement of a sugar phosphate unit from the sugar phosphate backbone by another unit,
d) the replacement of a β-D-ribose unit by a modified sugar unit, and
e) the replacement of a natural nucleotide base by a modified nucleotide base.

More detailed examples for the chemical modification of an oligonucleotide are as follows.

A phosphodiester internucleotide bridge located at the 3′ and/or the 5′ end of a nucleotide can be replaced by a modified internucleotide bridge, wherein the modified internucleotide bridge is for example selected from phosphorothioate, phosphorodithioate, NR¹R²-phosphoramidate, boranophosphate, α-hydroxybenzyl phosphonate, phosphate-(C₁-C₂₁)-O-alkyl ester, phosphate-[(C₆-C₁₂)aryl-(C₁-C₂₁)-O-alkyl]ester, (C₁-C₈)alkylphosphonate and/or (C₆-C₁₂)arylphosphonate bridges, (C₇-C₁₂)-α-hydroxymethyl-aryl (e.g., disclosed in WO 95/01363), wherein (C₆-C₁₂)aryl, (C₆-C₂₀)aryl and (C₆-C₁₄)aryl are optionally substituted by halogen, alkyl, alkoxy, nitro, cyano, and where R¹ and R² are, independently of each other, hydrogen, (C₁-C₁₈)-alkyl, (C₆-C₂₀)-aryl, (C₆-C₁₄)-aryl-(C₁-C₈)-alkyl, preferably hydrogen, (C₁-C₈)-alkyl, preferably (C₁-C₄)-alkyl and/or methoxyethyl, or R¹ and R² form, together with the nitrogen atom carrying them, a 5-6-membered heterocyclic ring which can additionally contain a further heteroatom from the group O, S and N.

The replacement of a phosphodiester bridge located at the 3′ and/or the 5′ end of a nucleotide by a dephospho bridge (dephospho bridges are described, for example, in Uhlmann E and Peyman A in “Methods in Molecular Biology”, Vol. 20, “Protocols for Oligonucleotides and Analogs”, S. Agrawal, Ed., Humana Press, Totowa 1993, Chapter 16, pp. 355 ff), wherein a dephospho bridge is for example selected from the dephospho bridges formacetal, 3′-thioformacetal, methylhydroxylamine, oxime, methylenedimethyl-hydrazo, dimethylenesulfone and/or silyl groups.

A sugar phosphate unit (i.e., a β-D-ribose and phosphodiester internucleotide bridge together forming a sugar phosphate unit) from the sugar phosphate backbone (i.e., a sugar phosphate backbone is composed of sugar phosphate units) can be replaced by another unit, wherein the other unit is for example suitable to build up a “morpholino-derivative” oligomer (as described, for example, in Stirchak E P et al. (1989) Oligonucleotides Res 17:6129-41), that is, e.g., the replacement by a morpholino-derivative unit; or to build up a polyamide oligonucleotide (“PNA”; as described for example, in Nielsen P E et al. (1994) Bioconjug Chem 5:3-7), that is, e.g., the replacement by a PNA backbone unit, e.g., by 2-aminoethylglycine.

A β-ribose unit or a β-D-2′-deoxyribose unit can be replaced by a modified sugar unit, wherein the modified sugar unit is for example selected from β-D-ribose, α-D-2′-deoxyribose, L-2′-deoxyribose, 2′-F-2′-deoxyribose, 2′-F-arabinose, 2′-O—(C₁-C₆)alkyl-ribose, preferably 2′-O—(C₁-C₆)alkyl-ribose is 2′-O-methylribose, 2′-O—(C₂-C₆)alkenyl-ribose, 2′-[O—(C₁-C₆)alkyl-O—(C₁-C₆)alkyl]-ribose, 2′-NH₂-2′-deoxyribose, β-D-xylo-furanose, α-arabinofuranose, 2,4-dideoxy-β-D-erythro-hexo-pyranose, and carbocyclic (described, for example, in Froehler J (1992) Am Chem Soc 114:8320) and/or open-chain sugar analogs (described, for example, in Vandendriessche et al. (1993) Tetrahedron 49:7223) and/or bicyclosugar analogs (described, for example, in Tarkov M et al. (1993) Helv Chim Acta 76:481).

In some embodiments the sugar is 2′-O-methylribose, particularly for one or both nucleotides linked by a phosphodiester or phosphodiester-like internucleotide linkage.

Oligonucleotides also include substituted purines and pyrimidines such as C-5 propyne pyrimidine and 7-deaza-7-substituted purine modified bases. Wagner R W et al. (1996) Nat Biotechnol 14:840-4. Purines and pyrimidines include but are not limited to adenine, cytosine, guanine, and thymine, and other naturally and non-naturally occurring nucleobases, substituted and unsubstituted aromatic moieties.

A modified base is any base which is chemically distinct from the naturally occurring bases typically found in DNA and RNA such as T, C, G, A, and U, but which share basic chemical structures with these naturally occurring bases. The modified nucleotide base may be, for example, selected from hypoxanthine, uracil, dihydrouracil, pseudouracil, 2-thiouracil, 4-thiouracil, 5-aminouracil, 5-(C₁-C₆)-alkyluracil, 5-(C₂-C₆)-alkenyluracil, 5-(C₂-C₆)-alkynyluracil, 5-(hydroxymethyl)uracil, 5-chlorouracil, 5-fluorouracil, 5-bromouracil, 5-hydroxycytosine, 5-(C₁-C₆)-alkylcytosine, 5-(C₂-C₆)-alkenylcytosine, 5-(C₂-C₆)-alkynylcytosine, 5-chlorocytosine, 5-fluorocytosine, 5-bromocytosine, N²-dimethylguanine, 2,4-diamino-purine, 8-azapurine, a substituted 7-deazapurine, preferably 7-deaza-7-substituted and/or 7-deaza-8-substituted purine, 5-hydroxymethylcytosine, N4-alkylcytosine, e.g., N4-ethylcytosine, 5-hydroxydeoxycytidine, 5-hydroxymethyldeoxycytidine, N4-alkyldeoxycytidine, e.g., N4-ethyldeoxycytidine, 6-thiodeoxyguanosine, and deoxyribonucleotides of nitropyrrole, C5-propynylpyrimidine, and diaminopurine e.g., 2,6-diaminopurine, inosine, 5-methylcytosine, 2-aminopurine, 2-amino-6-chloropurine, hypoxanthine or other modifications of a natural nucleotide bases. This list is meant to be exemplary and is not to be interpreted to be limiting.

In particular formulas described herein a set of modified bases is defined. For instance the letter Y is used to refer to a nucleotide containing a cytosine or a modified cytosine. A modified cytosine as used herein is a naturally occurring or non-naturally occurring pyrimidine base analog of cytosine which can replace this base without impairing the immunostimulatory activity of the oligonucleotide. Modified cytosines include but are not limited to 5-substituted cytosines (e.g. 5-methyl-cytosine, 5-fluoro-cytosine, 5-chloro-cytosine, 5-bromo-cytosine, 5-iodo-cytosine, 5-hydroxy-cytosine, 5-hydroxymethyl-cytosine, 5-difluoromethyl-cytosine, and unsubstituted or substituted 5-alkynyl-cytosine), 6-substituted cytosines, N4-substituted cytosines (e.g. N4-ethyl-cytosine), 5-aza-cytosine, 2-mercapto-cytosine, isocytosine, pseudo-isocytosine, cytosine analogs with condensed ring systems (e.g. N,N′-propylene cytosine or phenoxazine), and uracil and its derivatives (e.g. 5-fluoro-uracil, 5-bromo-uracil, 5-bromovinyl-uracil, 4-thio-uracil, 5-hydroxy-uracil, 5-propynyl-uracil). Some of the preferred cytosines include 5-methyl-cytosine, 5-fluoro-cytosine, 5-hydroxy-cytosine, 5-hydroxymethyl-cytosine, and N4-ethyl-cytosine. In another embodiment of the invention, the cytosine base is substituted by a universal base (e.g. 3-nitropyrrole, P-base), an aromatic ring system (e.g. fluorobenzene or difluorobenzene) or a hydrogen atom (dSpacer).

The letter Z is used to refer to guanine or a modified guanine base. A modified guanine as used herein is a naturally occurring or non-naturally occurring purine base analog of guanine which can replace this base without impairing the immunostimulatory activity of the oligonucleotide. Modified guanines include but are not limited to 7-deazaguanine, 7-deaza-7-substituted guanine (such as 7-deaza-7-(C2-C6)alkynylguanine), 7-deaza-8-substituted guanine, hypoxanthine, N2-substituted guanines (e.g. N2-methyl-guanine), 5-amino-3-methyl-3H,6H-thiazolo [4,5-d]pyrimidine-2,7-dione, 2,6-diaminopurine, 2-aminopurine, purine, indole, adenine, substituted adenines (e.g. N6-methyl-adenine, 8-oxo-adenine) 8-substituted guanine (e.g. 8-hydroxyguanine and 8-bromoguanine), and 6-thioguanine. In another embodiment of the invention, the guanine base is substituted by a universal base (e.g. 4-methyl-indole, 5-nitro-indole, and K-base), an aromatic ring system (e.g. benzimidazole or dichloro-benzimidazole, 1-methyl-1H-[1,2,4]triazole-3-carboxylic acid amide) or a hydrogen atom (dSpacer).

The oligonucleotides may have one or more accessible 5′ ends. It is possible to create modified oligonucleotides having two such 5′ ends. This may be achieved, for instance by attaching two oligonucleotides through a 3′-3′ linkage to generate an oligonucleotide having one or two accessible 5′ ends. The 3′-3′-linkage may be a phosphodiester, phosphorothioate or any other modified internucleotide bridge. Methods for accomplishing such linkages are known in the art. For instance, such linkages have been described in Seliger, H.; et al., Oligonucleotide analogs with terminal 3′-3′- and 5′-5′-internucleotidic linkages as antisense inhibitors of viral gene expression, Nucleotides & Nucleotides (1991), 10(1-3), 469-77 and Jiang, et al., Pseudo-cyclic oligonucleotides: in vitro and in vivo properties, Bioorganic & Medicinal Chemistry (1999), 7(12), 2727-2735.

Additionally, 3′-3′-linked oligonucleotides where the linkage between the 3′-terminal nucleotides is not a phosphodiester, phosphorothioate or other modified bridge, can be prepared using an additional spacer, such as tri- or tetra-ethylenglycol phosphate moiety (Durand, M. et al, Triple-helix formation by an oligonucleotide containing one (dA) 12 and two (dT) 12 sequences bridged by two hexaethylene glycol chains, Biochemistry (1992), 31(38), 9197-204, U.S. Pat. No. 5,658,738, and U.S. Pat. No. 5,668,265). Alternatively, the non-nucleotidic linker may be derived from ethanediol, propanediol, or from an abasic deoxyribose (dSpacer) unit (Fontanel, Marie Laurence et al., Sterical recognition by T4 polynucleotide kinase of non-nucleosidic moieties 5′-attached to oligonucleotides; Oligonucleotides Research (1994), 22(11), 2022-7) using standard phosphoramidite chemistry. The non-nucleotidic linkers can be incorporated once or multiple times, or combined with each other allowing for any desirable distance between the 3′-ends of the two ODNs to be linked.

The oligonucleotides are partially resistant to degradation (e.g., are stabilized). A “stabilized oligonucleotide molecule” shall mean an oligonucleotide that is relatively resistant to in vivo degradation (e.g. via an exo- or endo-nuclease). Oligonucleotide stabilization can be accomplished via backbone modifications. Oligonucleotides having phosphorothioate linkages provide maximal activity and protect the oligonucleotide from degradation by intracellular exo- and endo-nucleases. Other modified oligonucleotides include phosphodiester modified oligonucleotides, combinations of phosphodiester and phosphorothioate oligonucleotide, methylphosphonate, methylphosphorothioate, phosphorodithioate, p-ethoxy, and combinations thereof.

Modified backbones such as phosphorothioates may be synthesized using automated techniques employing either phosphoramidate or H-phosphonate chemistries. Aryl-and alkyl-phosphonates can be made, e.g., as described in U.S. Pat. No. 4,469,863; and alkylphosphotriesters (in which the charged oxygen moiety is alkylated as described in U.S. Pat. No. 5,023,243 and European Patent No. 092,574) can be prepared by automated solid phase synthesis using commercially available reagents. Methods for making other DNA backbone modifications and substitutions have been described (e.g., Uhlmann, E. and Peyman, A., Chem. Rev. 90:544, 1990; Goodchild, J., Bioconjugate Chem. 1:165, 1990).

Other stabilized oligonucleotides include: nonionic DNA analogs, such as alkyl- and aryl-phosphates (in which the charged phosphonate oxygen is replaced by an alkyl or aryl group), phosphodiester and alkylphosphotriesters, in which the charged oxygen moiety is alkylated. Oligonucleotides which contain diol, such as tetraethyleneglycol or hexaethyleneglycol, at either or both termini have also been shown to be substantially resistant to nuclease degradation.

The immunostimulatory oligonucleotides may also contain one or more unusual linkages between the nucleotide or nucleotide-analogous moieties. The usual internucleoside linkage is a 3′5′-linkage. All other linkages are considered to be unusual internucleoside linkages, such as 2′5′-, 5′5′-, 3′3′-, 2′2′-, 2′3′-linkages. The nomenclature 2′ to 5′ is chosen according to the carbon atom of ribose. However, if unnatural sugar moieties are employed, such as ring-expanded sugar analogs (e.g. hexanose, cylohexene or pyranose) or bi- or tricyclic sugar analogs, then this nomenclature changes according to the nomenclature of the monomer. In 3′-deoxy-β-D-ribopyranose analogs (also called p-DNA), the mononucleotides are e.g. connected via a 4′2′-linkage.

If the oligonucleotide contains one 3′3′-linkage, then this oligonucleotide may have two unlinked 5′-ends. Similarly, if the oligonucleotide contains one 5′5′-linkage, then this oligonucleotide may have two unlinked 3′-ends. The accessibility of unlinked ends of nucleotides may be better accessible by their receptors. Both types of unusual linkages (3′3′- and 5′5′) were described by Ramalho Ortigao et al. (Antisense Research and Development (1992) 2, 129-46), whereby oligonucleotides having a 3′3′-linkage were reported to show enhanced stability towards cleavage by nucleases.

Different types of linkages can also be combined in one molecule which may lead to branching of the oligomer. If one part of the oligonucleotide is connected at the 3′-end via a 3′3′-linkage to a second oligonucleotide part and at the 2′-end via a 2′3′-linkage to a third part of the molecule, this results e.g. in a branched oligonucleotide with three 5′-ends (3′3′-, 2′3′-branched).

In principle, linkages between different parts of an oligonucleotide or between different oligonucleotides, respectively, can occur via all parts of the molecule, as long as this does not negatively interfere with the recognition by its receptor. According to the nature of the oligonucleotide, the linkage can involve the sugar moiety (Su), the heterocyclic nucleobase (Ba) or the phosphate backbone (Ph). Thus, linkages of the type Su-Su, Su-Ph, Su-Ba, Ba-Ba, Ba-Su, Ba-Ph, Ph-Ph, Ph-Su, and Ph-Ba are possible. If the oligonucleotides are further modified by certain non-nucleotidic substituents, the linkage can also occur via the modified parts of the oligonucleotides. These modifications also include modified oligonucleotides, e.g. PNA, LNA, or Morpholino Oligonucleotide analogs.

The linkages are preferably composed of C, H, N, O, S, B, P, and Halogen, containing 3 to 300 atoms. An example with 3 atoms is an acetal linkage (ODN1-3′-O—CH₂—O-3′-ODN2) connecting e.g. the 3′-hydroxy group of one nucleotide to the 3′-hydroxy group of a second oligonucleotide. An example with about 300 atoms is PEG-40 (tetraconta polyethyleneglycol). Preferred linkages are phosphodiester, phosphorothioate, methylphosphonate, phosphoramidate, boranophosphonate, amide, ether, thioether, acetal, thioacetal, urea, thiourea, sulfonamide, Schiff Base and disulfide linkages. It is also possible to use the Solulink BioConjugation System i.e., (www.trilinkbiotech.com).

If the oligonucleotide is composed of two or more sequence parts, these parts can be identical or different. Thus, in an oligonucleotide with a 3′3′-linkage, the sequences can be identical 5′-ODN1-3′3′-ODN1-5′ or different 5′-ODN1-3′3′-ODN2-5′. Furthermore, the chemical modification of the various oligonucleotide parts as well as the linker connecting them may be different. Since the uptake of short oligonucleotides appears to be less efficient than that of long oligonucleotides, linking of two or more short sequences results in improved immune stimulation. The length of the short oligonucleotides is preferably 2-20 nucleotides, more preferably 3-16 nucleotides, but most preferably 5-10 nucleotides. Preferred are linked oligonucleotides which have two or more unlinked 5′-ends.

The oligonucleotide partial sequences may also be linked by non-nucleotidic linkers, in particular abasic linkers (dSpacers), trietyhlene glycol units or hexaethylene glycol units. Further preferred linkers are alkylamino linkers, such as C3, C6, C12 aminolinkers, and also alkylthiol linkers, such as C3 or C6 thiol linkers. The oligonucleotides can also be linked by aromatic residues which may be further substituted by alkyl or substituted alkyl groups. The oligonucleotides may also contain a Doubler or Trebler unit (www.glenres.com), in particular those oligonucleotides with a 3′3′-linkage. Branching of the oligonucleotides by multiple doubler, trebler, or other multiplier units leads to dendrimers which are a further embodiment of this invention. The oligonucleotides may also contain linker units resulting from peptide modifying reagents or oligonucleotide modifying reagents (www.glenres.com). Furthermore, it may contain one or more natural or unnatural amino acid residues which are connected by peptide (amide) linkages.

Another possibility for linking oligonucleotides is via crosslinking of the heterocyclic bases (Verma and Eckstein; Annu. Rev. Biochem. (1998) 67: 99-134; page 124). A linkage between the sugar moiety of one sequence part with the heterocyclic base of another sequence part (Iyer et al. Curr. Opin. Mol. Therapeutics (1999) 1: 344-358; page 352) may also be used.

The different oligonucleotides are synthesized by established methods and can be linked together on-line during solid-phase synthesis. Alternatively, they may be linked together post-synthesis of the individual partial sequences.

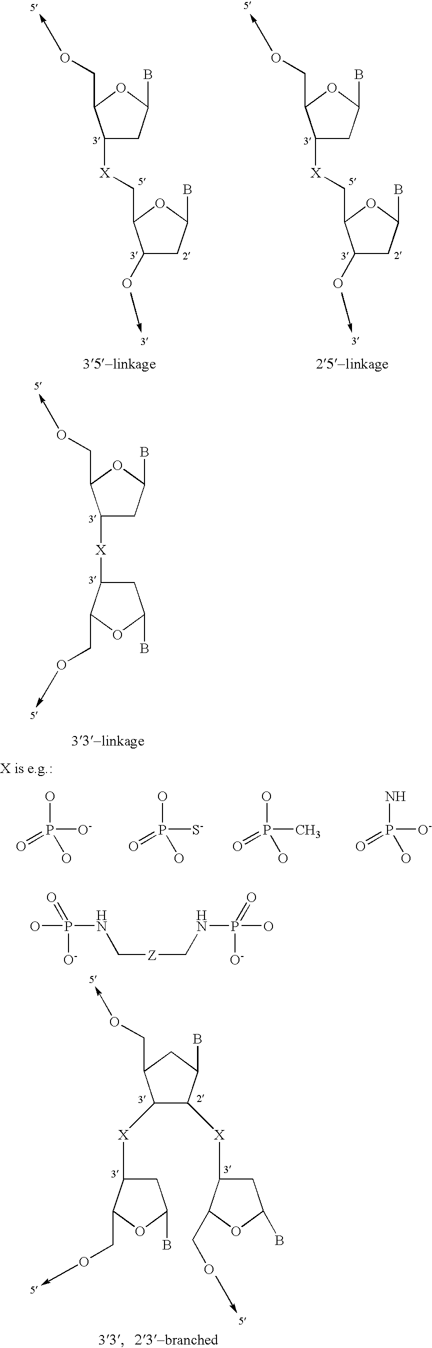

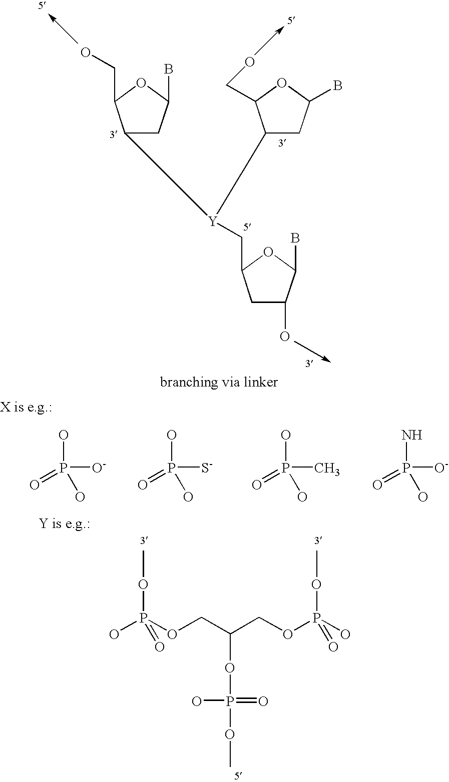

CpG immunostimulatory oligonucleotides can be combined with other therapeutic agents. The CpG immunostimulatory oligonucleotide and other therapeutic agent may be administered simultaneously or sequentially. When the other therapeutic agents are administered simultaneously they can be administered in the same or separate formulations, but are administered at the same time. The other therapeutic agents are administered sequentially with one another and with CpG immunostimulatory oligonucleotide, when the administration of the other therapeutic agents and the CpG immunostimulatory oligonucleotide is temporally separated. The separation in time between the administration of these compounds may be a matter of minutes or it may be longer. Other therapeutic agents include but are not limited to anti-microbials and anti asthma medicaments.

The oligonucleotides of the invention may be administered to a subject with an anti-microbial agent. An anti-microbial agent, as used herein, refers to a naturally-occurring or synthetic compound which is capable of killing or inhibiting infectious microorganisms. The type of anti-microbial agent useful according to the invention will depend upon the type of microorganism with which the subject is infected or at risk of becoming infected. Anti-microbial agents include but are not limited to anti-bacterial agents, anti-viral agents, anti-fungal agents and anti-parasitic agents. Phrases such as “anti-infective agent”, “anti-bacterial agent”, “anti-viral agent”, “anti-fungal agent”, “anti-parasitic agent” and “parasiticide” have well-established meanings to those of ordinary skill in the art and are defined in standard medical texts. Briefly, anti-bacterial agents kill or inhibit bacteria, and include antibiotics as well as other synthetic or natural compounds having similar functions. Antibiotics are low molecular weight molecules which are produced as secondary metabolites by cells, such as microorganisms. In general, antibiotics interfere with one or more bacterial functions or structures which are specific for the microorganism and which are not present in host cells. Anti-viral agents can be isolated from natural sources or synthesized and are useful for killing or inhibiting viruses. Anti-fungal agents are used to treat superficial fungal infections as well as opportunistic and primary systemic fungal infections. Anti-parasite agents kill or inhibit parasites.

Examples of anti-parasitic agents, also referred to as parasiticides useful for human administration include but are not limited to albendazole, amphotericin B, benznidazole, bithionol, chloroquine HCl, chloroquine phosphate, clindamycin, dehydroemetine, diethylcarbamazine, diloxamide furoate, eflomithine, furazolidaone, glucocorticoids, halofantrine, iodoquinol, ivermectin, mebendazole, mefloquine, meglumine antimoniate, melarsoprol, metrifonate, metronidazole, niclosamide, nifurtimox, oxamniquine, paromomycin, pentamidine isethionate, piperazine, praziquantel, primaquine phosphate, proguanil, pyrantel pamoate, pyrimethanmine-sulfonamides, pyrimethanmine-sulfadoxine, quinacrine HCl, quinine sulfate, quinidine gluconate, spiramycin, stibogluconate sodium (sodium antimony gluconate), suramin, tetracycline, doxycycline, thiabendazole, timidazole, trimethroprim-sulfamethoxazole, and tryparsamide some of which are used alone or in combination with others.

Antibacterial agents kill or inhibit the growth or function of bacteria. A large class of antibacterial agents is antibiotics. Antibiotics, which are effective for killing or inhibiting a wide range of bacteria, are referred to as broad spectrum antibiotics. Other types of antibiotics are predominantly effective against the bacteria of the class gram-positive or gram-negative. These types of antibiotics are referred to as narrow spectrum antibiotics. Other antibiotics which are effective against a single organism or disease and not against other types of bacteria, are referred to as limited spectrum antibiotics. Antibacterial agents are sometimes classified based on their primary mode of action. In general, antibacterial agents are cell wall synthesis inhibitors, cell membrane inhibitors, protein synthesis inhibitors, oligonucleotide synthesis or functional inhibitors, and competitive inhibitors.

Antiviral agents are compounds which prevent infection of cells by viruses or replication of the virus within the cell. There are many fewer antiviral drugs than antibacterial drugs because the process of viral replication is so closely related to DNA replication within the host cell, that non-specific antiviral agents would often be toxic to the host. There are several stages within the process of viral infection which can be blocked or inhibited by antiviral agents. These stages include, attachment of the virus to the host cell (immunoglobulin or binding peptides), uncoating of the virus (e.g. amantadine), synthesis or translation of viral mRNA (e.g. interferon), replication of viral RNA or DNA (e.g. nucleotide analogues), maturation of new virus proteins (e.g. protease inhibitors), and budding and release of the virus.

Nucleotide analogues are synthetic compounds which are similar to nucleotides, but which have an incomplete or abnormal deoxyribose or ribose group. Once the nucleotide analogues are in the cell, they are phosphorylated, producing the triphosphate formed which competes with normal nucleotides for incorporation into the viral DNA or RNA. Once the triphosphate form of the nucleotide analogue is incorporated into the growing oligonucleotide chain, it causes irreversible association with the viral polymerase and thus chain termination. Nucleotide analogues include, but are not limited to, acyclovir (used for the treatment of herpes simplex virus and varicella-zoster virus), gancyclovir (useful for the treatment of cytomegalovirus), idoxuridine, ribavirin (useful for the treatment of respiratory syncitial virus), dideoxyinosine, dideoxycytidine, zidovudine (azidothymidine), imiquimod, and resimiquimod.

The interferons are cytokines which are secreted by virus-infected cells as well as immune cells. The interferons function by binding to specific receptors on cells adjacent to the infected cells, causing the change in the cell which protects it from infection by the virus. α and β-interferon also induce the expression of Class I and Class II MHC molecules on the surface of infected cells, resulting in increased antigen presentation for host immune cell recognition. α and β-interferons are available as recombinant forms and have been used for the treatment of chronic hepatitis B and C infection. At the dosages which are effective for anti-viral therapy, interferons have severe side effects such as fever, malaise and weight loss.

Anti-viral agents useful in the invention include but are not limited to immunoglobulins, amantadine, interferons, nucleotide analogues, and protease inhibitors. Specific examples of anti-virals include but are not limited to Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscarnet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; and Zinviroxime.

Anti-fungal agents are useful for the treatment and prevention of infective fungi. Anti-fungal agents are sometimes classified by their mechanism of action. Some anti-fungal agents function as cell wall inhibitors by inhibiting glucose synthase. These include, but are not limited to, basiungin/ECB. Other anti-fungal agents function by destabilizing membrane integrity. These include, but are not limited to, immidazoles, such as clotrimazole, sertaconzole, fluconazole, itraconazole, ketoconazole, miconazole, and voriconacole, as well as FK 463, amphotericin B, BAY 38-9502, MK 991, pradimicin, UK 292, butenafine, and terbinafine. Other anti-fungal agents function by breaking down chitin (e.g. chitinase) or immunosuppression (501 cream).

An “asthma medicament” as used herein is a composition of matter which reduces the symptoms, inhibits the asthmatic reaction, or prevents the development of an asthmatic reaction. Various types of medicaments for the treatment of asthma are described in the Guidelines For The Diagnosis and Management of Asthma, Expert Panel Report 2, NIH Publication No. 97/4051, Jul. 19, 1997, the entire contents of which are incorporated herein by reference. The summary of the medicaments as described in the NIH publication is presented below.

Asthma medicaments include, but are not limited to, steroids, PDE-4 inhibitors, bronchodilator/beta-2 agonists, K+ channel openers, VLA-4 antagonists, neurokin antagonists, TXA2 synthesis inhibitors, xanthanines, arachidonic acid antagonists, 5 lipoxygenase inhibitors, thromboxin A2 receptor antagonists, thromboxane A2 antagonists, inhibitor of 5-lipox activation proteins, and protease inhibitors.

Bronchodilator/beta-2 agonists are a class of compounds which cause bronchodilation or smooth muscle relaxation. Bronchodilator/beta-2 agonists include, but are not limited to, salmeterol, salbutamol, albuterol, terbutaline, D2522/formoterol, fenoterol, bitolterol, pirbuerol methylxanthines and orciprenaline. Long-acting β₂ agonists and bronchodilators are compounds which are used for long-term prevention of symptoms in addition to the anti-inflammatory therapies. They function by causing bronchodilation, or smooth muscle relaxation, following adenylate cyclase activation and increase in cyclic AMP producing functional antagonism of bronchoconstriction. These compounds also inhibit mast cell mediator release, decrease vascular permeability and increase mucociliary clearance. Long-acting β2 agonists include, but are not limited to, salmeterol and albuterol. These compounds are usually used in combination with corticosteroids and generally are not used without any inflammatory therapy. They have been associated with side effects such as tachycardia, skeletal muscle tremor, hypokalemia, and prolongation of QTc interval in overdose.

Methylxanthines, including for instance theophylline, have been used for long-term control and prevention of symptoms. These compounds cause bronchodilation resulting from phosphodiesterase inhibition and likely adenosine antagonism. It is also believed that these compounds may effect eosinophilic infiltration into bronchial mucosa and decrease T-lymphocyte numbers in the epithelium. Dose-related acute toxicities are a particular problem with these types of compounds. As a result, routine serum concentration must be monitored in order to account for the toxicity and narrow therapeutic range arising from individual differences in metabolic clearance. Side effects include tachycardia, nausea and vomiting, tachyarrhythmias, central nervous system stimulation, headache, seizures, hematemesis, hyperglycemia and hypokalemia. Short-acting β₂ agonists/bronchodilators relax airway smooth muscle, causing the increase in air flow. These types of compounds are a preferred drug for the treatment of acute asthmatic systems. Previously, short-acting β₂ agonists had been prescribed on a regularly-scheduled basis in order to improve overall asthma symptoms. Later reports, however, suggested that regular use of this class of drugs produced significant diminution in asthma control and pulmonary function (Sears, et al. Lancet; 336:1391-6, 1990). Other studies showed that regular use of some types of β₂ agonists produced no harmful effects over a four-month period but also produced no demonstrable effects (Drazen, et al., N. Eng. J. Med.; 335:841-7, 1996). As a result of these studies, the daily use of short-acting β₂ agonists is not generally recommended. Short-acting β₂ agonists include, but are not limited to, albuterol, bitolterol, pirbuterol, and terbutaline. Some of the adverse effects associated with the mastration of short-acting β₂ agonists include tachycardia, skeletal muscle tremor, hypokalemia, increased lactic acid, headache, and hyperglycemia.

The CpG immunostimulatory oligonucleotides may be directly administered to the subject or may be administered in conjunction with a nucleic acid delivery complex. A nucleic acid delivery complex shall mean a nucleic acid molecule associated with (e.g. ionically or covalently bound to; or encapsulated within) a targeting means (e.g. a molecule that results in higher affinity binding to target cell. Examples of nucleic acid delivery complexes include oligonucleotides associated with a sterol (e.g. cholesterol), a lipid (e.g. a cationic lipid, virosome or liposome), or a target cell specific binding agent (e.g. a ligand recognized by target cell specific receptor). Preferred complexes may be sufficiently stable in vivo to prevent significant uncoupling prior to internalization by the target cell. However, the complex can be cleavable under appropriate conditions within the cell so that the nucleic acid is released in a functional form.

Delivery vehicles or delivery devices for delivering antigen and oligonucleotides to surfaces have been described. The CpG immunostimulatory oligonucleotide and/or the antigen and/or other therapeutics may be administered alone (e.g., in saline or buffer) or using any delivery vehicles known in the art. For instance the following delivery vehicles have been described: Cochleates; Emulsomes, ISCOMs; Liposomes; Live bacterial vectors (e.g., Salmonella, Escherichia coli, Bacillus calmatte-guerin, Shigella, Lactobacillus); Live viral vectors (e.g., Vaccinia, adenovirus, Herpes Simplex); Microspheres; Oligonucleotide vaccines; Polymers; Polymer rings; Proteosomes; Sodium Fluoride; Transgenic plants; Virosomes; Virus-like particles. Other delivery vehicles are known in the art and some additional examples are provided below in the discussion of vectors.

The term effective amount of a CpG immunostimulatory oligonucleotide refers to the amount necessary or sufficient to realize a desired biologic effect. For example, an effective amount is that amount sufficient to reduce or prevent further induction of viral load in order to avoid exacerbation of asthma. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular CpG immunostimulatory oligonucleotide being administered the size of the subject, or the severity of the disease or condition. One of ordinary skill in the art can empirically determine the effective amount of a particular CpG immunostimulatory oligonucleotide and/or other therapeutic agent without necessitating undue experimentation.

Subject doses of the compounds described herein for mucosal or local delivery typically range from about 0.1 μg to 10 mg per administration, which depending on the application could be given daily, weekly, or monthly and any other amount of time therebetween. More typically mucosal or local doses range from about 10 μg to 5 mg per administration, and most typically from about 100 μg to 1 mg, with 2-4 administrations being spaced days or weeks apart. More typically, immune stimulant doses range from 1 μg to 10 mg per administration, and most typically 10 μg to 1 mg, with daily or weekly administrations. Subject doses of the compounds described herein for parenteral delivery for the purpose of inducing an immune response may be typically 5 to 10,000 times higher than the effective mucosal dose, and more typically 10 to 1,000 times higher, and most typically 20 to 100 times higher. Doses of the compounds described herein for parenteral delivery for the purpose of inducing an innate immune response or for inducing an immune response when the CpG immunostimulatory oligonucleotides are administered in combination with other therapeutic agents or in specialized delivery vehicles typically range from about 0.1 μg to 10 mg per administration, which depending on the application could be given daily, weekly, or monthly and any other amount of time therebetween. More typically parenteral doses for these purposes range from about 10 μg to 5 mg per administration, and most typically from about 100 μg to 1 mg, with 2-4 administrations being spaced days or weeks apart. In some embodiments, however, parenteral doses for these purposes may be used in a range of 5 to 10,000 times higher than the typical doses described above. The oligonucleotides may be administered in multiple doses over extended period of time.

For any compound described herein the therapeutically effective amount can be initially determined from animal models. A therapeutically effective dose can also be determined from human data for CpG oligonucleotides which have been tested in humans (human clinical trials have been initiated) and for compounds which are known to exhibit similar pharmacological activities, such as other adjuvants, e.g., LT and other antigens for vaccination purposes. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the CpG immunostimulatory oligonucleotide can be administered to a subject by any mode that delivers the oligonucleotide to the desired surface, e.g., mucosal, systemic. Administering the pharmaceutical composition of the present invention may be accomplished by any means known to the skilled artisan. Preferred routes of administration include but are not limited to oral, parenteral, intramuscular, intranasal, sublingual, intratracheal, inhalation, ocular, vaginal, and rectal.

For oral administration, the compounds (i.e., CpG immunostimulatory oligonucleotides and other therapeutic agents) can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, i.e. EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189. Other polymers that could be used are poly-1,3-dioxolane and poly-1,3,6-tioxocane. Preferred for pharmaceutical usage, as indicated above, are polyethylene glycol moieties.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the oligonucleotide (or derivative) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e. powder; for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the oligonucleotide (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, a-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the oligonucleotide or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Also contemplated herein is pulmonary delivery of the oligonucleotides (or derivatives thereof). The oligonucleotide (or derivative) is delivered to the lungs of a mammal while inhaling and traverses across the lung epithelial lining to the blood stream. Other reports of inhaled molecules include Adjei et al., 1990, Pharmaceutical Research, 7:565-569; Adjei et al., 1990, International Journal of Pharmaceutics, 63:135-144 (leuprolide acetate); Braquet et al., 1989, Journal of Cardiovascular Pharmacology, 13(suppl. 5):143-146 (endothelin-1); Hubbard et al., 1989, Annals of Internal Medicine, Vol. III, pp. 206-212 (a1-antitrypsin); Smith et al., 1989, J. Clin. Invest. 84:1145-1146 (a-1-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J. Immunol. 140:3482-3488 (interferon-g and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of oligonucleotide (or derivative). Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified oligonucleotide may also be prepared in different formulations depending on the type of chemical modification or the type of device employed.

Formulations suitable for use with a nebulizer, either jet or ultrasonic, will typically comprise oligonucleotide (or derivative) dissolved in water at a concentration of about 0.1 to 25 mg of biologically active oligonucleotide per mL of solution. The formulation may also include a buffer and a simple sugar (e.g., for oligonucleotide stabilization and regulation of osmotic pressure). The nebulizer formulation may also contain a surfactant, to reduce or prevent surface induced aggregation of the oligonucleotide caused by atomization of the solution in forming the aerosol.

Formulations for use with a metered-dose inhaler device will generally comprise a finely divided powder containing the oligonucleotide (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing oligonucleotide (or derivative) and may also include a bulking agent, such as lactose, sorbitol, sucrose, or mannitol in amounts which facilitate dispersal of the powder from the device, e.g., 50 to 90% by weight of the formulation. The oligonucleotide (or derivative) should most advantageously be prepared in particulate form with an average particle size of less than 10 mm (or microns), most preferably 0.5 to 5 mm, for most effective delivery to the distal lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The CpG immunostimulatory oligonucleotides and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

### Example 1

1. Induction of IFNα and IFN-Associated Genes by a C-class CpG ODN (SEQ ID NO:10)

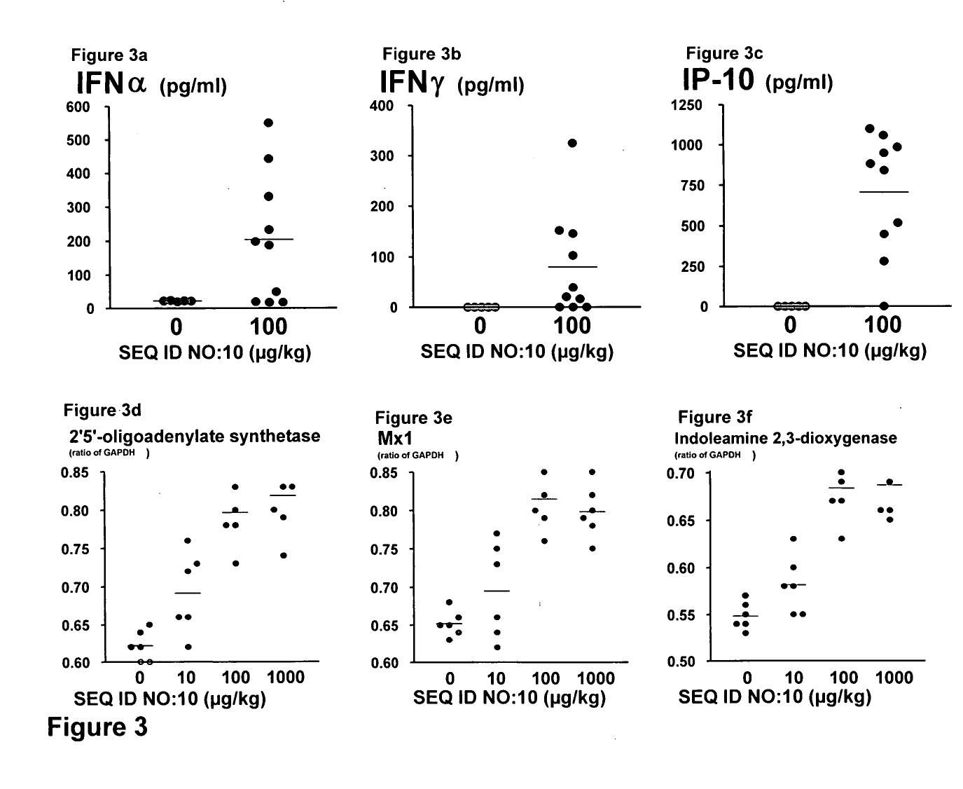

Methods: Mice (male, BALB/c) received SEQ ID NO:10 (100 μg/kg in FIGS. 3a-3c or 10, 100, or 1000 μl/kg in FIGS. 3d-3f) or saline by intranasal instillation. Secreted proteins (IFN α, IFN γ, and IP10) were assayed in bronchoalveolar lavage fluid 15 hours later, or gene expression in lung tissue was analyzed by real-time PCR 30 hours later.

Results: C-class CpG ODN induced secretion of IFNα, IFNγ and interferon-inducible protein-10 (IP-10). The results are shown in FIG. 3.

Since the CpG ODN stimulated secretion of IFNα in mouse airways, we investigated whether the interferon-inducible gene for indoleamine 2,3 dioxygenase becomes expressed in the lung. When instilled in the airways, the CpG ODN did increase expression of mRNA for this immune-modulating enzyme (FIG. 3f). Mx1, and indoleamine 2,3-dioxygenase were also upregulated in mouse lung (FIGS. 3d and 3e).

2. Anti-Viral Effects of a C-class CpG ODN

Because respiratory tract virus infections are a major cause of asthma exacerbations, a mouse model was established in which airway inflammation is exacerbated by combined antigen challenge and virus infection.

Methods Mice received two adminsitrations of SEQ ID NO:10 at 30, 100, or 300 μg/kg, 4 days apart, by intranasal instillation in 40 μl saline. Two days after the last dose, mice were infected with influenza virus (influenza type A, subtype H1N1, mouse adapted strain PR8, 200 EID50, in 40 μl saline) by intranasal instillation.

Virus load in the lung (Takara Biomedical enzyme immunoassay for nuclear protein) and airway inflammation (counts of cells recovered by bronchoalveolar lavage) were assessed 6 days after virus infection.

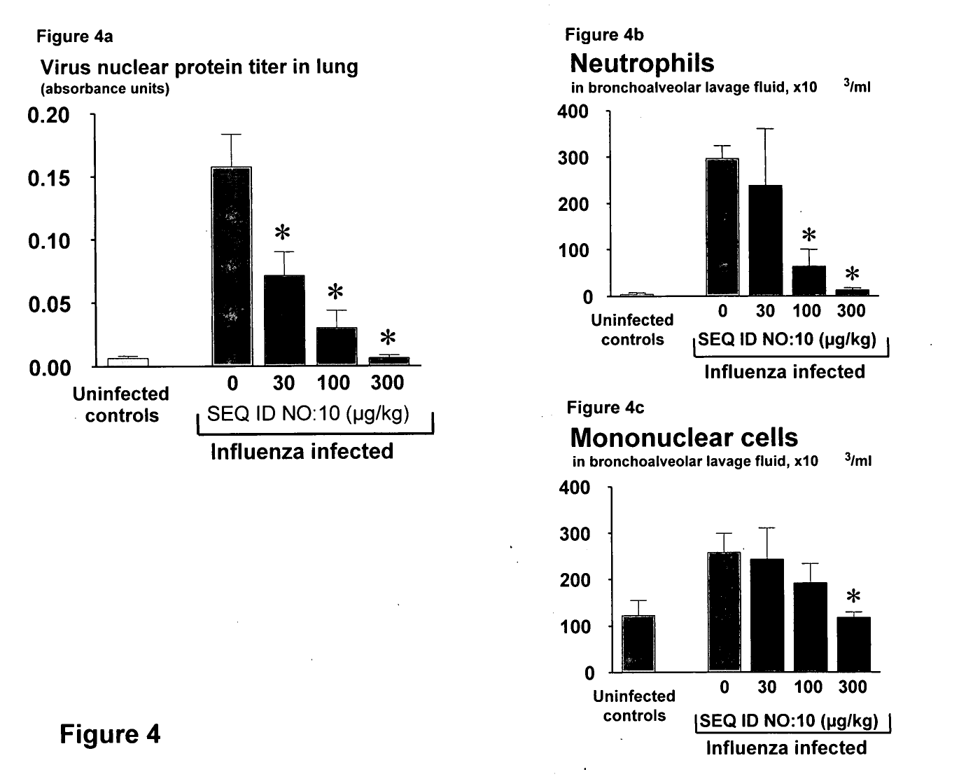

Results: Pretreatment with SEQ ID NO: 10 reduced influenza virus load in the lung (FIG. 4a) and virus-induced accumulation of leukocytes (including neutrophils and mononuclear cells) in the airways (FIGS. 4b and 4c). The results are shown in FIG. 4.

3. Protective Effects of a C-Class CpG ODN Against Antigen- and Virus-Induced Airway Inflammation and Hyperreactivty

Methods Mice were sensitized with antigen (cockroach, 10 μg, intraperitoneal with aluminum hydroxide adjuvant) and then challenged twice each week for three weeks with intranasal antigen (10 μg in 40 μl saline). Mice were infected with influenza virus by intranasal instillation before the last pair of antigen challenges. Alternatively, separate mice received antigen challenge alone or virus infection alone.

SEQ ID NO:10 (100 μg/kg) was administered intranasally once each week, two days before the first antigen challenge of the week. Airway inflammation (counts of cells recovered by bronchoalveolar lavage) and airway hyperreactivity to inhaled methacholine (Sigma, St. Louis, Mo., USA) were assessed 48 hours after the last antigen challenge. Mice were anaesthetized with sodium pentobarbitone (60 mg/kg, intraperitoneal) and mechanically ventilated through a tracheal cannula. Cells were recovered from the airways by bronchoalveolar lavage performed with 1 ml of RPMI 1640 medium containing 10% fetal bovine serum (both from Invitrogen, Carlsbad, Calif., USA) instilled through a tracheal cannula. Airway resistance was calculated from measurements of pulmonary airflow and intratracheal pressure using respiratory mechanics software (Buxco Research Systems, Wilmington, N.C., USA). After recording baseline airway resistance, increasing concentrations of methacholine aerosol (5-100 mg/ml for 5 seconds, at 5 minute intervals) were delivered through the tracheal cannula. The resulting bronchoconstriction was measured as increase in airway resistance. For each animal, the area under the methacholine dose-response curve was calculated.

Analysis of data: Statistical significance of differences between treatment group and untreated control group means were determined using the Mann-Whitney test or Kruskal-Wallis multiple comparison test (* P<0.05).

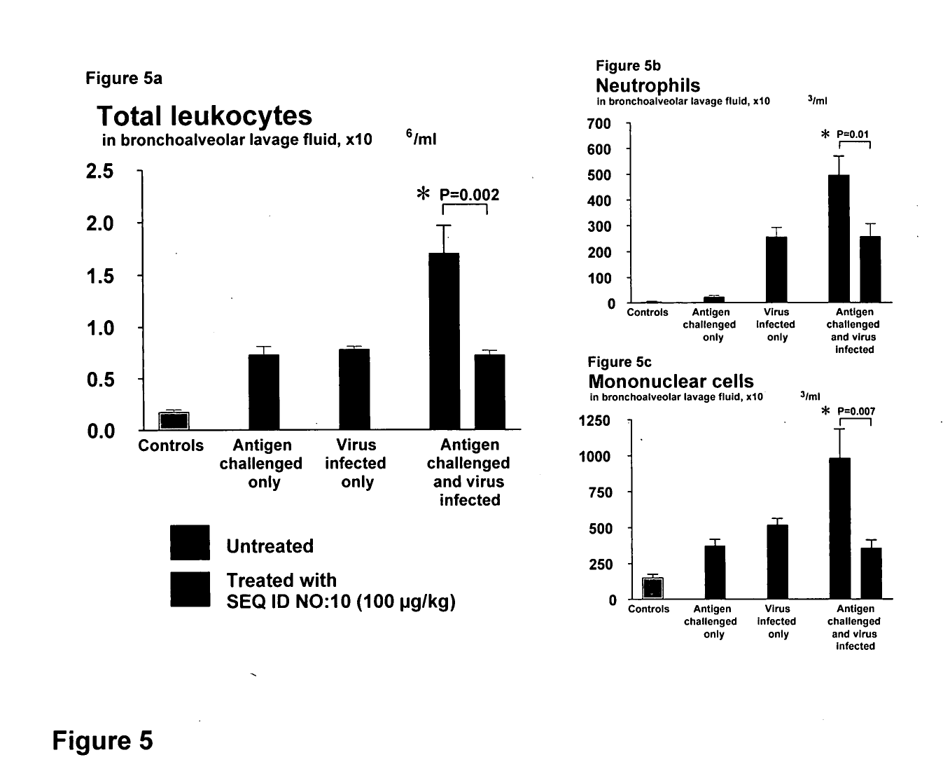

Results: Mice that were both antigen-challenged and virus-infected showed a more severe accumulation of leukocytes (including neutrophils and mononuclear cells) in the airways than mice that were either antigen challenged alone or virus infected alone (FIGS. 5a-5c).

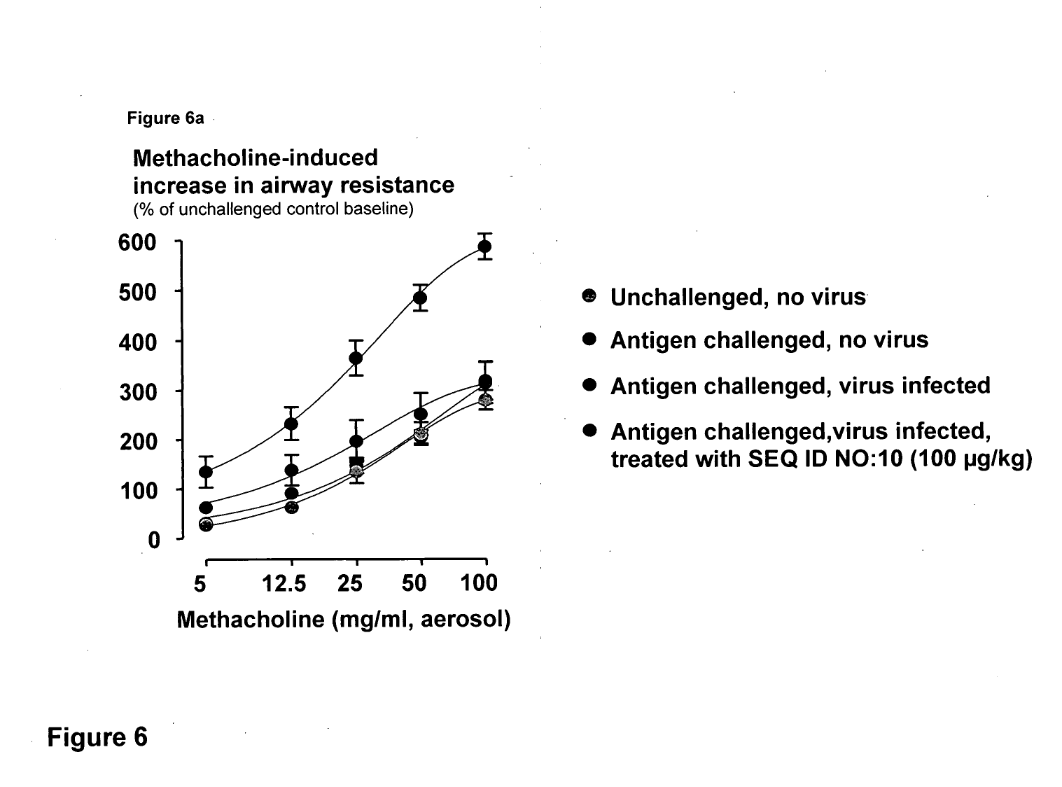

These mice also developed airway hyperreactivity. When dosed into the airways once each week for three weeks, the CpG ODN protected mice against the exacerbated airways inflammation and the fall in body weight, and almost completely prevented the increase in baseline airway resistance and the development of airway hyperreactivity (FIGS. 6a-6c).

### Example 2

It has been demonstrated that the class C CpG oligodeoxynucleotide can suppress influenza virus load and virus-induced airway inflammation in mice. In Example 2 the protective effects of SEQ ID NO: 10 against the exacerbated airway inflammation induced by combined influenza virus infection and antigen challenge were examined.

Methods

1. Antigen and Virus Administrations:

Mice (male BALB/c) were sensitized on study days 0 and 7 with antigen (cockroach, 10 μg, intraperitoneal) with aluminum hydroxide adjuvant (Pierce Alum).

Mice were antigen challenged by exposure to intranasally-administered antigen (10 μg in 40 μl saline), twice each week for three consecutive weeks. The first challenge was on study day 21.

Mice were infected with influenza virus (influenza type A, subtype H1N1, mouse adapted strain PR8, 200 EID₅₀ in 40 μl saline) by intranasal instillation on study day 34 (i.e. before the last pair of antigen challenges).

Alternatively, separate groups of mice received antigen challenge alone or virus infection alone.

2. Treatment with SEQ ID NO:10:

SEQ ID NO:10 (100 μg/kg) was administered intranasally once each week, two days before the first antigen challenge of the week.

3. Endpoints:

Airway inflammation was assessed 48 hours after the last antigen challenge. Cells in airways were recovered by bronchoalveolar lavage. Differential cell counts were made by light microscopy from cytocentrifuge preparations stained with Wright-Giemza stain.

Summary of Study Protocol

TABLE 2

| | | | | | | | | | | Virus | | | |
|---|---|---|---|---|---|---|---|---|---|---|---|---|---|
| | | | | | | | | | | ↓ | | | |
| | Antigen | | | Antigen | | | Antigen | | | | Antigen | | |
| | sensitize | | | challenge | | | challenge | | | | challenge | | |
| | ↓ | ↓ | | ↓ | ↓ | | ↓ | ↓ | | | ↓ | ↓ | |
| | | | ODN | | | ODN | | | ODN | | | | |
| | | | ↓ | | | ↓ | | | ↓ | | | | |
| Day: | 0 | 7 | 19 | 21 | 24 | 26 | 28 | 31 | 33 | 34 | 35 | 38 | 40 |
| | | | | | | | | | | | | | ↓ |
| | | | | | | | | | | | | | Endpoints |

| | | |
|---|---|---|
| First | Second | Third |
| treatment week | treatment week | treatment week |

Results

Characterization of Virus- and Antigen-Induced Airway Inflammation

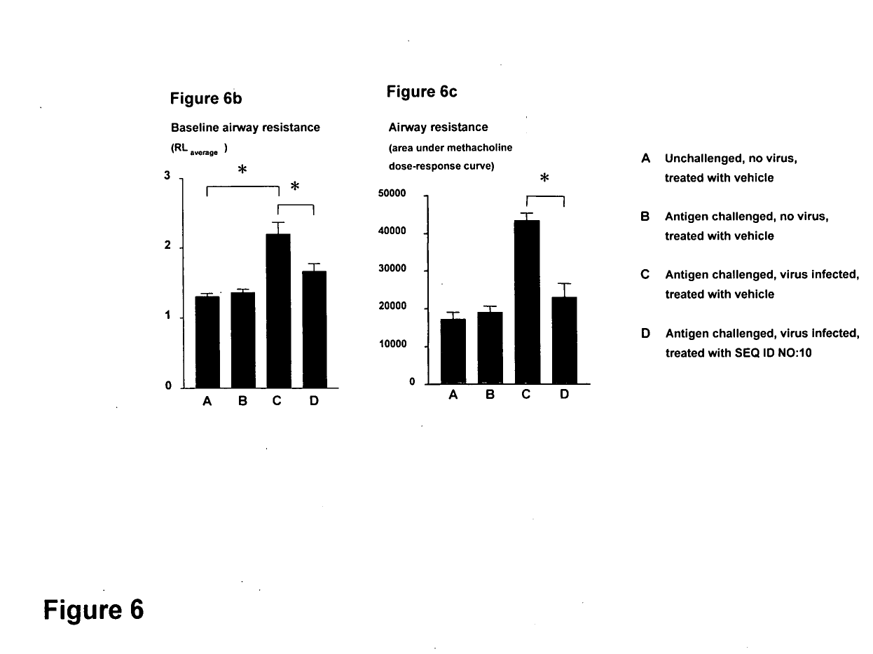

Infection with influenza virus alone or antigen challenge alone each caused an increase in the total number of leukocytes in bronchoalveolar lavage fluid (FIG. 7). In virus-infected mice, this cell accumulation included a marked neutrophilia, whereas in antigen-challenged mice, the accumulation included a marked eosinophilia.

When compared with mice that received antigen challenge alone, those that were antigen-challenged and virus-infected showed an exacerbated accumulation of leukocytes in bronchoalveolar lavage fluid (FIG. 7). This increased accumulation included both neutrophils and mononuclear cells. However, these mice showed reduced eosinophilia.

Effects of SEQ ID NO: 10:

Treatment with SEQ ID NO:10 (100 μg/kg) did not suppress the virus-induced neutrophilia (FIG. 7). This finding was expected at this dose. It has been determined that a higher dose of 300 μg/kg generally demonstrates better anti-virus effects.

In contrast, SEQ ID NO: 10 (100 μg/kg) significantly suppressed antigen-induced cellular infiltration (FIG. 7).

An important finding of this study was that SEQ ID NO:10 (100 μg/kg) significantly suppressed the exacerbated airway inflammation induced in mice that were both virus-infected and antigen-challenged. The exacerbated accumulations of neutrophils and mononuclear cells were both suppressed (FIG. 7).

In addition to exacerbated airway inflammation, mice that were both virus-infected and antigen-challenged showed a marked loss of body weight. This was significantly suppressed in mice treated with SEQ ID NO: 10.

### Example 3

Induction of TLR9-Associated Cytokines from Mouse Splenocytes In Vitro, and in the Mouse Lung In Vivo

The ability of SEQ ID NO: 10 to induce secretion of TLR9-associated cytokines from murine splenocytes in vitro was examined.

Methods

Stimulation of Cytokines from Splenocytes In Vitro

Splenocytes were pooled from 6 mice and incubated with ODNs (0.1, 1 or 10 μg/ml) for 36 hours. Cells were isolated mechanically by gently pushing chopped mouse spleens through a cell sieve (70 μm pore size). Cells (1×10⁷, pooled from 6 mice) were incubated (37° C., 5% CO₂) in 1 ml medium (RPMI 1640 containing 10% fetal bovine serum, both from Invitrogen, Carlsbad, Calif., USA). SEQ ID NO: 10 or control ODN (with reversed CpG motifs) or either of the two domains of SEQ ID NO: 10 (5′ end stimulatory sequence and palindrome) were added to give concentrations of 0.1, 1 or 10 μg/ml. After incubation for 24 hours, culture medium was assayed as described below for secreted cytokines (IFNα, IFNγ, interferon-inducible protein [IP]-10, IL-6, IL-10 and TNFα).

Stimulation of Cytokines in Mouse Airways

Mice received SEQ ID NO:10 (10-1000 μg/kg) or vehicle (40 μl saline) delivered into the airways by intranasal instillation carried out under light isoflurane anaesthesia. Twenty-four hours later, bronchoalveolar lavage was performed through a tracheal cannula using 1 ml of saline. Cytokine concentrations (IFNα, IFNγ, IP-10, IL-6 and IL-12p40) in bronchoalveloar lavage fluid were assayed.

Results

As shown in table one, SEQ ID NO: 10 induced secretion of TLR9-associated cytokines from isolated murine splenocytes. In contrast, a control ODN with reversed CpG motifs, the 5′ end stimulatory sequence of SEQ ID NO: 10 alone, or the palindrome alone had no marked activity. The highest titers of each cytokine were induced with ODNs at 10 μg/ml (data from lower concentrations are not shown). n.d.=not detected (<12 pg/ml). Thus, SEQ ID NO: 10 induced secretions of IFNα, IFNγ, IP-10, IL-6, IL-10 and TNFα in a concentration-dependent manner. The highest titers of each cytokine were induced with SEQ ID NO: 10 at 10 μg/ml.

To evaluate the importance of correctly-ordered CpG motifs for this biological activity of SEQ ID NO:10, the assay was repeated with an ODN with the same sequence as SEQ ID NO:10 but with reversed CpG motifs in the 5′ end stimulatory sequence (SEQ ID NO: 55). The control oligonucleotide showed almost no ability to induce these TLR9-associated cytokines. The 5′ end stimulatory sequence of SEQ ID NO:10 alone, or the palindrome alone, also had no marked activity demonstrating that an intact molecule with both these domains is required for activity (sequences shown in Table 3).

TABLE 3

| | Cytokine titer (pg/ml) induced by ODN | | | | | |
|---|---|---|---|---|---|---|
| | (10 μg/ml) | | | | | |
| ODN | IFNα | IFNγ | IP-10 | IL-6 | IL-10 | TNFα |
| Medium alone | 34.4 | n.d. | 21.9 | 64.8 | n.d. | n.d. |
| SEQ ID NO: 10 | 199.0 | 539.9 | 111.1 | 11531.5 | 113.1 | 71.1 |
| Control ODN | 22.1 | n.d. | 22.4 | 537.5 | n.d. | n.d. |
| Stimulatory | 19.5 | n.d. | 17.9 | 427.1 | n.d. | 21.0 |
| sequence alone | | | | | | |
| Palindrome alone | 18.8 | n.d. | 17.7 | 59.0 | n.d. | n.d. |

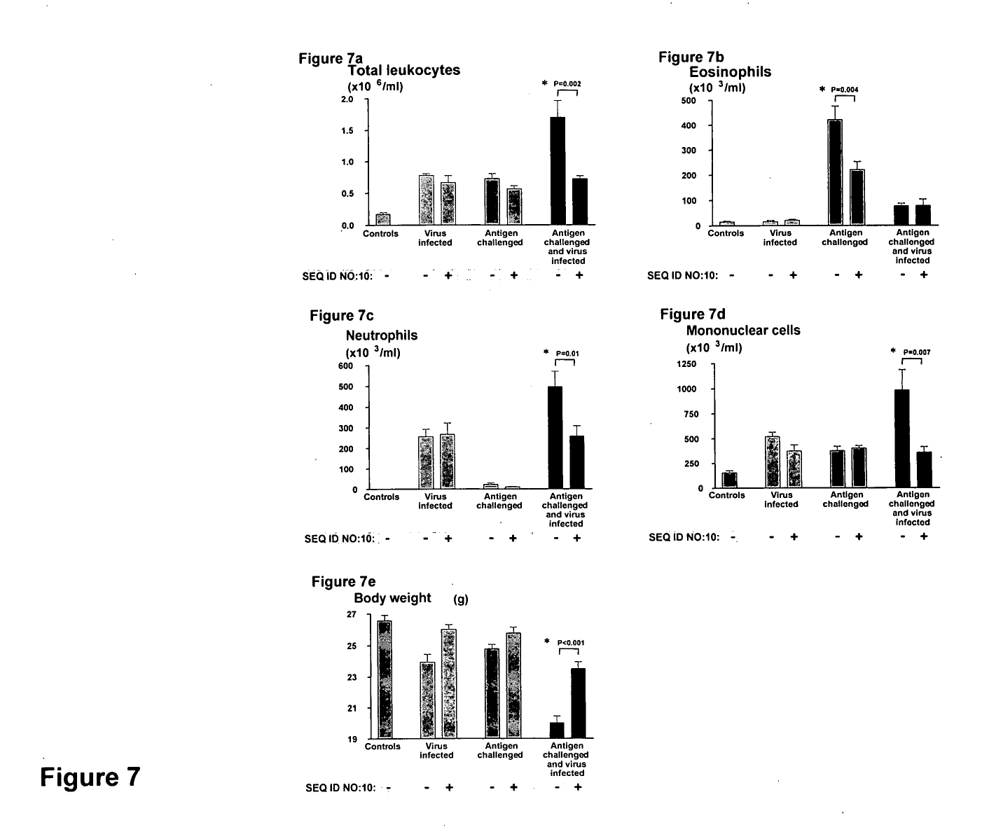

It was then investigated whether SEQ ID NO:10 could induce TLR9-associated cytokines when dosed into mouse airways in vivo. SEQ ID NO: 10 induced secretion of IFNαc, IFNγ, IP-10, IL-6 and IL-12p40 as demonstrated by increased concentrations of these cytokines in bronchoalveolar lavage fluid (FIG. 8).

### Example 4

SEQ ID NO: 10 Induces Immune Deviation Away from a Th2 Response to Antigen Sensitization.

To determine whether SEQ ID NO: 10 could suppress a Th2 response to antigen sensitization when injected into the mouse footpad together with a sensitizing antigen (ovalbumin), the mice were re-stimulated with antigen in a recall assay ex vivo.

Methods

Mice were sensitized with antigen (10 μg grade V ovalbumin, Sigma, St. Louis, Mo., USA) injected into the right rear footpad. Antigen was injected either alone, or together with SEQ ID NO:10 (10-1000 μg/kg). In each case, total injection volume was 20 μl. Six days later, the draining popliteal lymph node was removed and a cell suspension was prepared by gently pushing the nodes through a cell sieve (70 μm pore size). An antigen recall assay was carried out ex vivo by incubating (37° C., 5% CO₂) 1×10⁶ unfractionated lymph node cells in 220 μl medium (RPMI 1640 containing 10% fetal bovine serum, both from Invitrogen, Carlsbad, Calif., USA) in the presence or absence of antigen (ovalbumin, 10 μg/ml). After incubation for 36 hours, culture medium was assayed as described below for secreted cytokines (IL-5, IL-13 and IFNγ).

Results

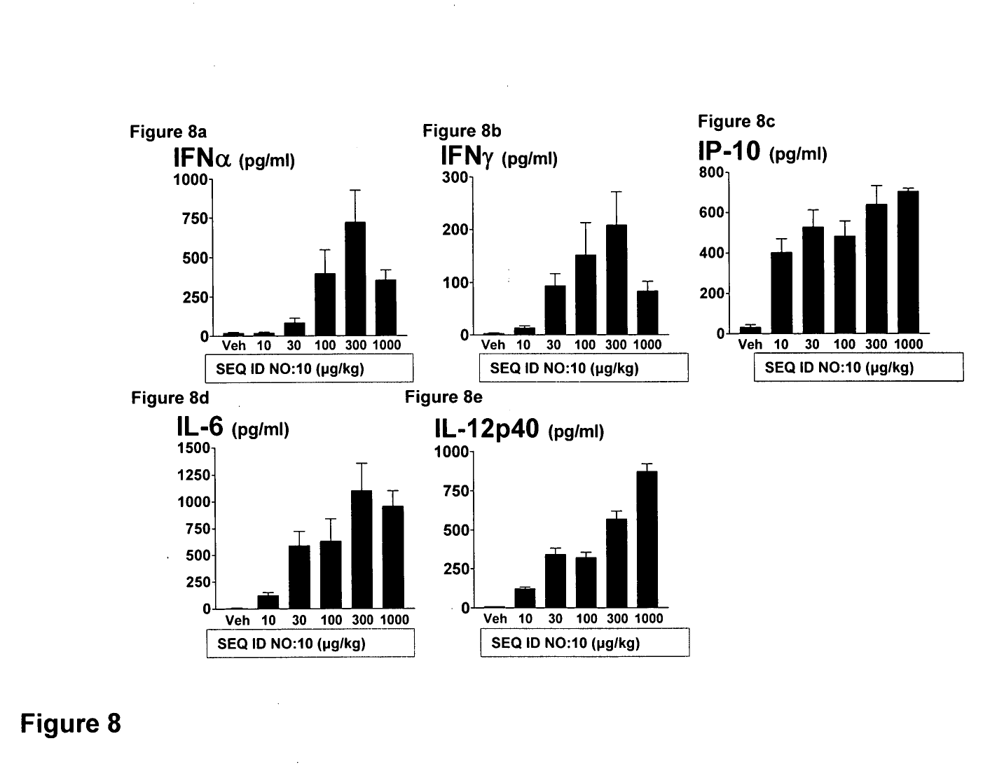

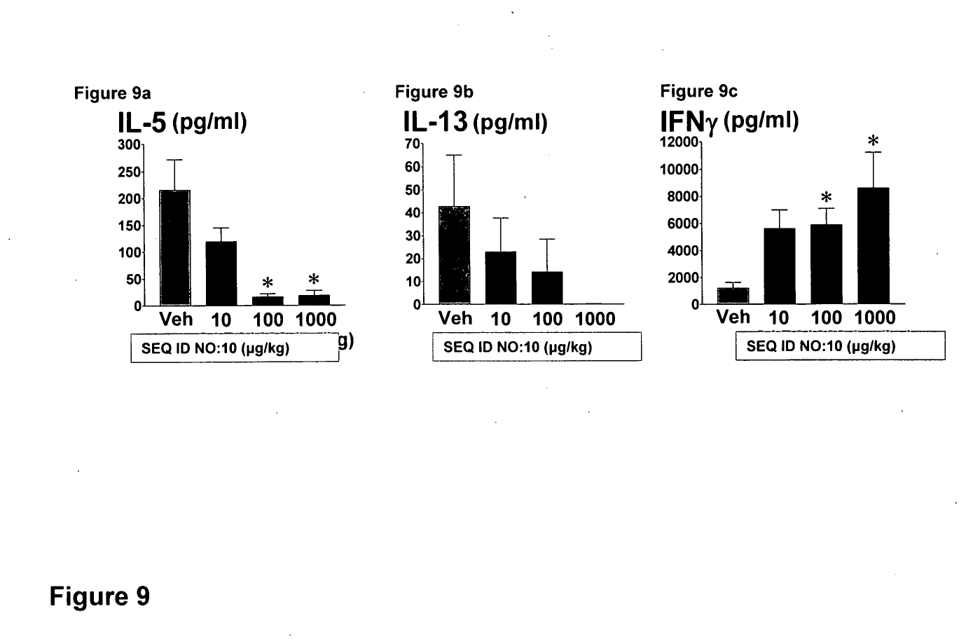

Popliteal lymph node cells from sensitized mice secreted IL-5, IL-13 and IFNγ (FIG. 9). Cells incubated in the absence of antigen, or with a control antigen (cockroach) to which the mice had not been sensitized, did not secrete detectable titers of any of these cytokines (<19 pg/ml). Cells isolated from SEQ ID NO: 10-treated animals showed attenuated antigen-induced secretions of the Th2 cytokines IL-5 and IL-13. In contrast, secretion of the Th1 cytokine IFNγ was markedly increased (FIG. 9c). Cells incubated in the absence of antigen, or with a control antigen (cockroach) to which the mice had not been sensitized, did not secrete detectable titers of any of these cytokines (<10 pg/ml).

### Example 5

SEQ ID NO: 10 Suppresses Antigen-Induced IgE Production and Stimulates IgG2a Production in the Mouse In Vivo.

It was next determined whether SEQ ID NO: 10 could alter the profile of immunoglobulin production when dosed to mice at the time of antigen sensitization.

Methods

Mice were antigen sensitized twice, 7 days apart, with intraperitoneal antigen (10 μg grade V ovalbumin, Sigma, St. Louis, Mo., USA) dissolved in aluminum hydroxide adjuvant (0.2 ml, Pierce Imject Alum, Rockford, Ill., USA). Mice received SEQ ID NO:10 (1-1000 μg/kg) or control vehicle (saline, 10 ml/kg) by intraperitoneal injection two days before each of the two sensitizations, and on the day of each sensitization. Mice were bled by cardiac puncture 12 days after the second sensitization. Serum was collected by centrifugation and assayed as follows for ovalbumin-specific IgE and IgG2a.

ELISAs were carried out in microtiter plates (Nunc, Rochester, N.Y., USA), with washes using 0.05% polysorbate 20 (Sigma, St. Louis, Mo., USA) in phosphate buffered saline (Invitrogen, Carlsbad, Calif., USA) between each of the following steps. Plates were coated with ovalbumin (150 μl of 100 μg/ml) in binding buffer (0.1M NaHCO₃ Sigma) for 15 hours at 4° C. Plates were then blocked with assay diluent (200 μl/well, Pharmingen, BD Biosciences, Franklin Lakes, N.J., USA) for 2 hours at 20° C. Serum samples (diluted 1 in 40 in assay diluent, 100 μl/well) were added and left for 2 hours at 20° C. Biotin-conjugated rat anti-mouse IgE or IgG2a (Pharmingen) (2 μg/ml in assay diluent, 100 μl/well) were added and left for 2 hours at 4° C. Streptavidin-conjugated horseradish peroxidase (Pharmingen, diluted 1:1000 in assay diluent, 100 μl/well) was then added and left for 1 hour at 20° C. Tetramethyl benzidine substrate reagent (Pharmingen, 100 μl/well) was added for 30 minutes at 20° C. and the reaction was then stopped with 2N sulphuric acid (50 μl/well). Absorbance at 450 nm was measured using a spectrophotometer (Spectramax, Molecular Devices, Sunnyvale, Calif., USA).

Results

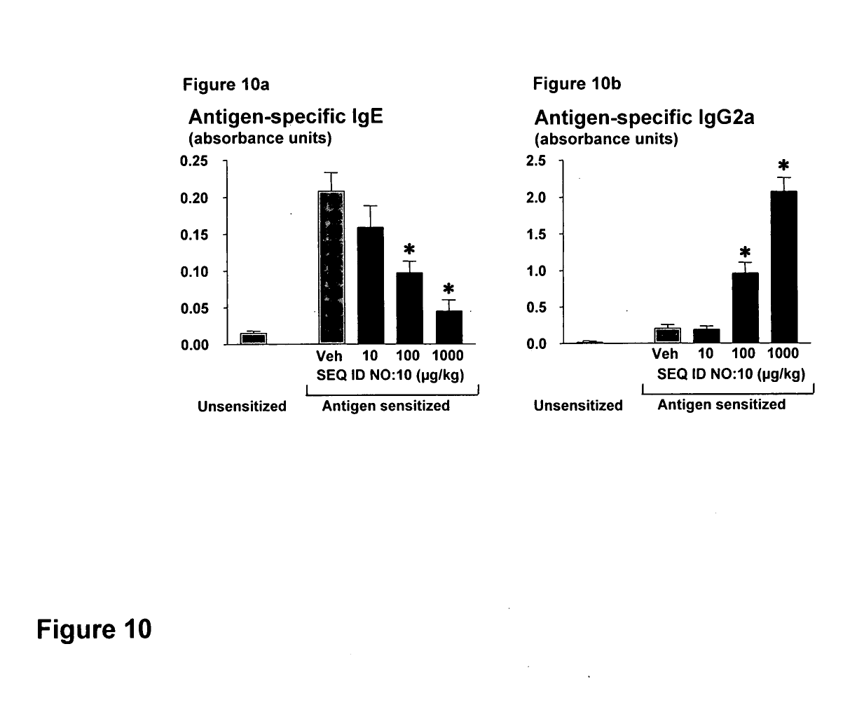

SEQ ID NO:10 suppressed the production of antigen-specific IgE (85% suppression with a dose of 1000 μg/kg) and potentiated the production of IgG2a providing further evidence of immune deviation away from a Th2 response to antigen (FIG. 10).

### Example 6

SEQ ID NO: 10 Suppresses Antigen-Induced Accumulations of Eosinophils and Lymphocytes in Mouse Airways In Vivo.

Examples 4 and 5 demonstrate that SEQ ID NO: 10 is able to suppress Th2 immune responses. Therefore, the protective effects of SEQ ID NO: 10 in a mouse model of antigen-induced airway inflammation were examined.

Methods

Mice were sensitized with intraperitoneal antigen (cockroach) and then antigen challenged twice a week for 2 weeks with antigen instilled into the airways. During each of the 2 challenge weeks, mice were treated once with SEQ ID NO:10 or vehicle (Veh) instilled into the airways. Alternatively, mice were untreated (Untr). Bronchoalveolar lavage was performed 48 hours after the last antigen challenge and recovered cells were counted. Total leukocytes (a) and eosinophils (b) were counted with an automated cell counter.

Results

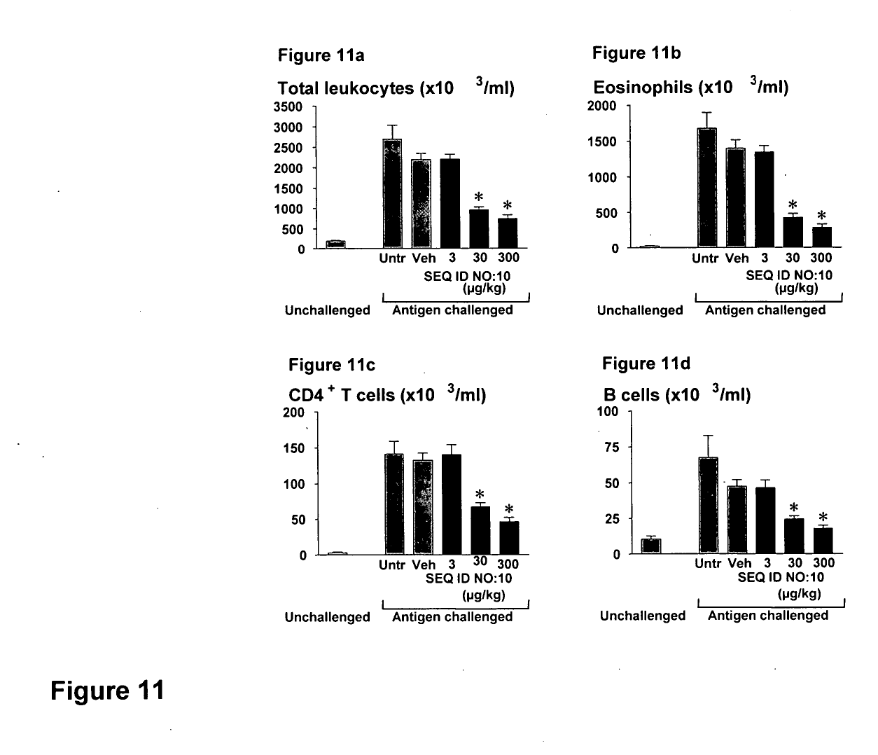

As this experimental model shares hallmark features of allergic asthma, the protective effects of SEQ ID NO:10 for this indication were examined When dosed into the airways once each week for two weeks, SEQ ID NO:10 suppressed the airway accumulations of eosinophils, T cells and B cells that were induced by intrapulmonary antigen challenge (FIG. 11). At the highest dose tested (300 μg/kg), SEQ ID NO:10 suppressed accumulations of these cells by 78%, 65% and 79% respectively.

### CONCLUSIONS

In both children and adults with existing asthma, infections with respiratory tract viruses are important precipitants for airway obstruction and wheezing. The inflammatory processes involved are complex. However, virus-induced neutrophil and mononuclear cell recruitment and activation are implicated in aggravating the airway obstruction that contributes to these asthma exacerbations. The data presented herein demonstrate that CpG ODN, particularly C-class ODN, markedly suppress the exacerbated accumulations of neutrophils and mononuclear cells induced in mice by combined virus infection and antigen challenge.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

What is claimed is:

1. A method for treating viral exacerbated asthma, comprising:
administering to an asthmatic subject an effective amount of a C-class CpG oligonucleotide for treating viral exacerbated asthma.

2. The method of claim 1, wherein the viral exacerbated asthma is caused by a respiratory virus.

3. The method of claim 2, wherein the respiratory virus is not RSV.

4. The method of claim 1, wherein the viral exacerbated asthma is caused by influenza virus.

5. The method of claim 1, wherein the subject is identified by a medical worker.

6. The method of claim 1, wherein the subject is identified based on exposure to a risk factor for viral infection.

7. The method of claim 1, wherein the method includes the step of identifying an asthmatic subject at risk of viral infection.

8. The method of claim 1, wherein the C-class oligonucleotide is a semi-soft oligonucleotide.

9. The method of claim 1, wherein the C-class oligonucleotide is SEQ ID NO: 10.

10. A method for treating viral exacerbated asthma, comprising:
identifying an asthmatic subject at risk of viral infection, and
administering to the asthmatic subject an effective amount of a CpG oligonucleotide for treating viral exacerbated asthma.

11. The method of claim 10, wherein the viral exacerbated asthma is caused by a respiratory virus.

12. The method of claim 11, wherein the respiratory virus is not RSV.

13. The method of claim 10, wherein the viral exacerbated asthma is caused by influenza virus.

14. The method of claim 10, wherein the risk factor is influenza season.

15. The method of claim 10, wherein the risk factor is travel to a destination with a high risk of viral exposure.

16. The method of claim 10, wherein the CpG oligonucleotide is a C-class oligonucleotide.

17. The method of claim 16, wherein the C-class oligonucleotide is a semi-soft oligonucleotide.

18. The method of claim 16, wherein the C-class oligonucleotide is SEQ ID NO:10.

19. A method for treating viral exacerbated asthma, comprising:
administering to an asthmatic subject undergoing a non-CpG asthma therapy an effective amount of a CpG oligonucleotide for treating viral exacerbated asthma.

20. The method of claim 19, wherein the non-CpG asthma therapy is steroid therapy.

21. The method of claim 19, wherein the non-CpG asthma therapy is administered at a different time than the CpG oligonucleotide.

22. The method of claim 19, wherein the non-CpG asthma therapy is administered at the same time as the CpG oligonucleotide.

23. The method of claim 19, wherein the CpG oligonucleotide is a C-class oligonucleotide.

24. The method of claim 23, wherein the C-class oligonucleotide is a semi-soft oligonucleotide.

25. The method of claim 23, wherein the C-class oligonucleotide is SEQ ID NO:10.

26. A method for treating infectious disease exacerbated asthma, comprising:
identifying an asthmatic subject at risk of infection, and
administering to the asthmatic subject an effective amount of a CpG oligonucleotide for treating infectious disease exacerbated asthma.

27. A method for treating viral exacerbated asthma, comprising:
identifying an asthmatic subject at risk of viral infection, and
administering to the asthmatic subject a CpG oligonucleotide in an amount that is sub-therapeutic for treating viral infection, wherein the CpG oligonucleotide is effective for reducing immune cell accumulation.

28. The method of claim 27, wherein the immune cell is a neutrophil.

29. The method of claim 27, wherein the immune cell is an eosinophil

30. A method for treating viral exacerbated asthma, comprising:
identifying an asthmatic subject at risk of viral infection, and
administering to the asthmatic subject at least three doses of CpG oligonucleotide, wherein the at least three doses of CpG oligonucleotide are temporally separated from one another by at least three days.

31. A method for treating viral exacerbated asthma, comprising:
identifying a risk factor for viral infection, and
administering to an asthmatic subject an effective amount of a CpG oligonucleotide for treating viral exacerbated asthma during a period of time when the asthmatic subject is at risk of viral infection.


SEQUENCE LISTING (56 sequences)

SEQ ID NO: 1 - DNA, 22 bp, Artificial sequence
  Synthetic oligonucleotide
  misc_feature (1)..(2): wherein the internucleotide linkage is a stablized internucleotide linkage
  misc_feature (3)..(4): wherein the internucleotide linkage is a stablized internucleotide linkage
  misc_feature (5)..(7): wherein the internucleotide linkages are stablized internucleotide linkages
  misc_feature (8)..(11): wherein the internucleotide linkages are stablized internucleotide linkages
  misc_feature (12)..(16): wherein the internucleotide linkages are stablized internucleotide linkages
  misc_feature (17)..(22): wherein the internucleotide linkages are stablized internucleotide linkages
tcgcgtcgttcggcgcgcgccg

SEQ ID NO: 2 - DNA, 23 bp, Artificial sequence
  Synthetic oligonucleotide
  misc_feature (1)..(2): wherein the internucleotide linkage is a stabilized internucleotide linkage
  misc_feature (3)..(5): wherein the internucleotide linkages are stabilized internucleotide linkages
  misc_feature (6)..(8): wherein the internucleotide linkages are stabilized internucleotide linkages
  misc_feature (9)..(12): wherein the internucleotide linkages are stabilized internucleotide linkages
  misc_feature (13)..(17): wherein the internucleotide linkages are stabilized internucleotide linkages
  misc_feature (18)..(23): wherein the internucleotide linkages are stabilized internucleotide linkages
tcgtcgacgttcggcgcgcgccg

SEQ ID NO: 3 - DNA, 21 bp, Artificial sequence
  Synthetic oligonucleotide
  misc_feature (1)..(2): wherein the internucleotide linkage is a stabilized internucleotide linkage
  misc_feature (3)..(6): wherein the internucleotide linkages are stabilized internucleotide linkages
  misc_feature (7)..(10): wherein the internucleotide linkages are stabilized internucleotide linkages
  misc_feature (11)..(15): wherein the internucleotide linkages are stabilized internucleotide linkages
  misc_feature (16)..(21): wherein the internucleotide linkages are stabilized internucleotide linkages
tcggacgttcggcgcgcgccg

SEQ ID NO: 4 - DNA, 19 bp, Artificial sequence
  Synthetic oligonucleotide
  misc_feature (1)..(2): wherein the internucleotide linkage is a stabilized internucleotide linkage
  misc_feature (3)..(6): wherein the internucleotide linkages are stabilized internucleotide linkages
  misc_feature (7)..(10): wherein the internucleotide linkages are stabilized internucleotide linkages
  misc_feature (11)..(19): wherein the internucleotide linkages are stabilized internucleotide linkages
tcggacgttcggcgcgccg

SEQ ID NO: 5 - DNA, 20 bp, Artificial sequence
  Synthetic oligonucleotide
  misc_feature (1)..(2): wherein the internucleotide linkage is a stabilized internucleotide linkage
  misc_feature (3)..(4): wherein the internucleotide linkage is a stabilized internucleotide linkage
  misc_feature (5)..(7): wherein the internucleotide linkage is a stabilized internucleotide linkage
  misc_feature (8)..(11): wherein the internucleotide linkage is a stabilized internucleotide linkage
  misc_feature (12)..(20): wherein the internucleotide linkage is a stabilized internucleotide linkage
tcgcgtcgttcggcgcgccg

SEQ ID NO: 6 - DNA, 20 bp, Artificial sequence
  Synthetic oligonucleotide
  misc_feature (1)..(2): wherein the internucleotide linkage is a stabilized internucleotide linkage
  misc_feature (3)..(5): wherein the internucleotide linkages are stabilized internucleotide linkages
  misc_feature (6)..(9): wherein the internucleotide linkages are stabilized internucleotide linkages
  misc_feature (10)..(14): wherein the internucleotide linkages are stabilized internucleotide linkages
  misc_feature (15)..(20): wherein the internucleotide linkages are stabilized internucleotide linkages
tcgacgttcggcgcgcgccg

SEQ ID NO: 7 - DNA, 18 bp, Artificial sequence
  Synthetic oligonucleotide
  misc_feature (1)..(2): wherein the internucleotide linkage is a stabilized internucleotide linkage
  misc_feature (3)..(5): wherein the internucleotide linkages are stabilized internucleotide linkages
  misc_feature (6)..(9): wherein the internucleotide linkages are stabilized internucleotide linkages
  misc_feature (7)..(18): wherein the internucleotide linkages are stabilized internucleotide linkages
tcgacgttcggcgcgccg

SEQ ID NO: 8 - DNA, 18 bp, Artificial sequence
  Synthetic oligonucleotide
  misc_feature (1)..(2): wherein the internucleotide linkage is a stabilized internucleotide linkage
  misc_feature (3)..(4): wherein the internucleotide linkage is a stabilized internucleotide linkage
  misc_feature (5)..(7): wherein the internucleotide linkages are stabilized internucleotide linkages
  misc_feature (8)..(11): wherein the internucleotide linkages are stabilized internucleotide linkages
  misc_feature (12)..(18): wherein the internucleotide linkages are stabilized internucleotide linkages
tcgcgtcgttcggcgccg

SEQ ID NO: 9 - DNA, 22 bp, Artificial sequence
  Synthetic oligonucleotide
  misc_feature (1)..(2): wherein the internucleotide linkage is a stabilized internucleotide linkage
  misc_feature (3)..(4): wherein the internucleotide linkage is a stabilized internucleotide linkage
  misc_feature (5)..(7): wherein the internucleotide linkages are stabilized internucleotide linkages
  misc_feature (8)..(11): wherein the internucleotide linkages are stabilized internucleotide linkages
  misc_feature (12)..(16): wherein the internucleotide linkages are stabilized internucleotide linkages
  misc_feature (17)..(22): wherein the internucleotide linkages are stabilized internucleotide linkages
tcgcgacgttcggcgcgcgccg

SEQ ID NO: 10 - DNA, 23 bp, Artificial sequence
  Synthetic oligonucleotide
  misc_feature (1)..(2): wherein the internucleotide linkage is a stabilized internucleotide linkage
  misc_feature (3)..(5): wherein the internucleotide linkages are stabilized internucleotide linkages
  misc_feature (6)..(8): wherein the internucleotide linkages are stabilized internucleotide linkages
  misc_feature (9)..(12): wherein the internucleotide linkages are stabilized internucleotide linkages
  misc_feature (13)..(17): wherein the internucleotide linkages are stabilized internucleotide linkages
  misc_feature (18)..(23): wherein the internucleotide linkages are stabilized internucleotide linkages
tcgtcgtcgttcggcgcgcgccg

SEQ ID NO: 11 - DNA, 24 bp, Artificial sequence
  Synthetic oligonucleotide
  misc_feature (1)..(24): wherein the internucleotide linkages are stabilized internucleotide linkages
tcgtcgttttgacgttttgtcgtt

SEQ ID NO: 12 - DNA, 24 bp, Artificial sequence
  Synthetic oligonucleotide
  misc_feature (1)..(24): wherein the internucleotide linkages are stabilized internucleotide linkages
tcgtcgttttgtcgttttgtcgtt

SEQ ID NO: 13 - DNA, 24 bp, Artificial sequence
  Synthetic oligonucleotide
  misc_feature (1)..(5): wherein the internucleotide linkages are stabilized internucleotide linkages
  misc_feature (6)..(12): wherein the internucleotide linkages are stabilized internucleotide linkages
  misc_feature (13)..(18): wherein the internucleotide linkages are stabilized internucleotide linkages
  misc_feature (19)..(24): wherein the internucleotide linkages are stabilized internucleotide linkages
tcgtcgttttacggcgccgtgccg

SEQ ID NO: 14 - DNA, 24 bp, Artificial sequence
  Synthetic oligonucleotide
  misc_feature (1)..(2): wherein the internucleotide linkage is a stabilized internucleotide linkage
  misc_feature (3)..(5): wherein the internucleotide linkages are stabilized internucleotide linkages
  misc_feature (6)..(13): wherein the internucleotide linkages are stabilized internucleotide linkages
  misc_feature (14)..(21): wherein the internucleotide linkages are stabilized internucleotide linkages
  misc_feature (22)..(24): wherein the internucleotide linkages are stabilized internucleotide linkages
tcgtcgttttgacgttttgtcgtt

SEQ ID NO: 15 - DNA, 23 bp, Artificial sequence
  Synthetic oligonucleotide
  misc_feature (1)..(2): wherein the internucleotide linkage is a stabilized internucleotide linkage
  misc_feature (3)..(5): wherein the internucleotide linkages are stabilized internucleotide linkages
  misc_feature (6)..(8): wherein the internucleotide linkages are stabilized internucleotide linkages
  misc_feature (9)..(12): wherein the internucleotide linkages are stabilized internucleotide linkages
  misc_feature (13)..(17): wherein the internucleotide linkages are stabilized internucleotide linkages
  misc_feature (18)..(23): wherein the internucleotide linkages are stabilized internucleotide linkages
tcgtcgacgttcggcgcgcgccg

SEQ ID NO: 16 - DNA, 15 bp, Artificial sequence
  Synthetic oligonucleotide
  misc_feature (1)..(2): wherein the internucleotide linkage is a stabilized internucleotide linkage
  misc_feature (12)..(15): wherein the internucleotide linkage is a stabilized internucleotide linkage
tcgacgtcgtggggg

SEQ ID NO: 17 - DNA, 19 bp, Artificial sequence
  Synthetic oligonucleotide
  misc_feature (1)..(19): wherein the internucleotide linkages are stabilized internucleotide linkages
tcgacgtcgacgtgacgtg

SEQ ID NO: 18 - DNA, 17 bp, Artificial sequence
  Synthetic oligonucleotide
  misc_feature (1)..(17): wherein the internucleotide linkages are stabilized internucleotide linkages
tcgacgtcgacgtgacg

SEQ ID NO: 19 - DNA, 17 bp, Artificial sequence
  Synthetic oligonucleotide
  misc_feature (1)..(3): wherein the internucleotide linkages are stabilized internucleotide linkages
  misc_feature (4)..(6): wherein the internucleotide linkages are stabilized internucleotide linkages
  misc_feature (7)..(11): wherein the internucleotide linkages are stabilized internucleotide linkages
  misc_feature (12)..(14): wherein the internucleotide linkages are stabilized internucleotide linkages
  misc_feature (15)..(17): wherein the internucleotide linkages are stabilized internucleotide linkages
ttcgtcgttttgtcgtt

SEQ ID NO: 20 - DNA, 18 bp, Artificial sequence
  Synthetic oligonucleotide
  misc_feature (1)..(4): wherein the internucleotide linkages are stabilized internucleotide linkages
  misc_feature (5)..(7): wherein the internucleotide linkages are stabilized internucleotide linkages
  misc_feature (8)..(12): wherein the internucleotide linkages are stabilized internucleotide linkages
  misc_feature (13)..(15): wherein the internucleotide linkages are stabilized internucleotide linkages
  misc_feature (16)..(18): wherein the internucleotide linkages are stabilized internucleotide linkages
tttcgtcgtttcgtcgtt

SEQ ID NO: 21 - DNA, 21 bp, Artificial sequence
  Synthetic oligonucleotide
  misc_feature (1)..(5): wherein the internucleotide linkages are stabilized internucleotide linkages
  misc_feature (6)..(9): wherein the internucleotide linkages are stabilized internucleotide linkages
  misc_feature (10)..(15): wherein the internucleotide linkages are stabilized internucleotide linkages
  misc_feature (16)..(21): wherein the internucleotide linkages are stabilized internucleotide linkages
tcgtcgtacggcgccgtgccg

SEQ ID NO: 22 - DNA, 22 bp, Artificial sequence
  Synthetic oligonucleotide
  misc_feature (1)..(5): wherein the internucleotide linkages are stabilized internucleotide linkages
  misc_feature (6)..(10): wherein the internucleotide linkages are stabilized internucleotide linkages
  misc_feature (11)..(16): wherein the internucleotide linkages are stabilized internucleotide linkages
  misc_feature (17)..(22): wherein the internucleotide linkages are stabilized internucleotide linkages
tcgtcgttacggcgccgtgccg

SEQ ID NO: 23 - DNA, 19 bp, Artificial sequence
  Synthetic oligonucleotide
  misc_feature (1)..(19): wherein the internucleotide linkages are stabilized internucleotide linkages
tcgacgtcgacgtgacgtt

SEQ ID NO: 24 - DNA, 24 bp, Artificial sequence
  Synthetic oligonucleotide
  misc_feature (1)..(5): wherein the internucleotide linkages are stabilized internucleotide linkages
  misc_feature (6)..(8): wherein the internucleotide linkages are stabilized internucleotide linkages
  misc_feature (9)..(12): wherein the internucleotide linkages are stabilized internucleotide linkages
  misc_feature (13)..(18): wherein the internucleotide linkages are stabilized internucleotide linkages
  misc_feature (19)..(24): wherein the internucleotide linkages are stabilized internucleotide linkages
tcgtcgacgatcggcgccgtgccg

SEQ ID NO: 25 - DNA, 24 bp, Artificial sequence
  Synthetic oligonucleotide
  misc_feature (1)..(10): wherein the internucleotide linkages are stabilized internucleotide linkages
  misc_feature (12)..(24): wherein the internucleotide linkages are stabilized internucleotide linkages
tcgtcgacgatcggcgccgtgccg

SEQ ID NO: 26 - DNA, 19 bp, Artificial sequence
  Synthetic oligonucleotide
  misc_feature (1)..(5): wherein the internucleotide linkages are stabilized internucleotide linkages
  misc_feature (6)..(11): wherein the internucleotide linkages are stabilized internucleotide linkages
  misc_feature (12)..(19): wherein the internucleotide linkages are stabilized internucleotide linkages
tcgacgtcgacgtgacgtt

SEQ ID NO: 27 - DNA, 19 bp, Artificial sequence
  Synthetic oligonucleotide
  misc_feature (1)..(5): wherein the internucleotide linkages are stabilized internucleotide linkages
  misc_feature (6)..(13): wherein the internucleotide linkages are stabilized internucleotide linkages
  misc_feature (14)..(19): wherein the internucleotide linkages are stabilized internucleotide linkages
tcgacgtcgacgtgacgtt

SEQ ID NO: 28 - DNA, 24 bp, Artificial sequence
  Synthetic oligonucleotide
  misc_feature (1)..(5): wherein the internucleotide linkages are stabilized internucleotide linkages
  misc_feature (6)..(11): wherein the internucleotide linkages are stabilized internucleotide linkages
  misc_feature (12)..(17): wherein the internucleotide linkages are stabilized internucleotide linkages
  misc_feature (18)..(24): wherein the internucleotide linkages are stabilized internucleotide linkages
tcgtcgtttacggcgccgtgccgt

SEQ ID NO: 29 - DNA, 24 bp, Artificial sequence
  Synthetic oligonucleotide
  misc_feature (1)..(2): where the internucleotide linkage is a phosphorothioate internucleotide linkage
  misc_feature (3)..(5): where the internucleotide linkages are phosphorothioate internucleotide linkages
  misc_feature (6)..(13): where the internucleotide linkages are phosphorothioate internucleotide linkages
  misc_feature (14)..(21): where the internucleotide linkages are phosphorothioate internucleotide linkages
  misc_feature (22)..(24): where the internucleotide linkages are phosphorothioate internucleotide linkages
tcgtcgttttgacgttttgtcgtt

SEQ ID NO: 30 - DNA, 21 bp, Artificial sequence
  Synthetic oligonucleotide
  misc_feature (1)..(2): where the internucleotide linkage is a phosphorothioate internucleotide linkage
  misc_feature (3)..(10): where the internucleotide linkage is a phosphorothioate internucleotide linkage
  misc_feature (11)..(18): where the internucleotide linkage is a phosphorothioate internucleotide linkage
  misc_feature (19)..(21): where the internucleotide linkage is a phosphorothioate internucleotide linkage
tcgttttgacgttttgtcgtt

SEQ ID NO: 31 - DNA, 13 bp, Artificial sequence
  Synthetic oligonucleotide
  misc_feature (1)..(2): where the internucleotide linkage is a phosphorothioate internucleotide linkage
  misc_feature (3)..(10): where the internucleotide linkages are phosphorothioate internucleotide linkages
  misc_feature (11)..(13): where the internucleotide linkages are phosphorothioate internucleotide linkages
tcgttttgacgtt

SEQ ID NO: 32 - DNA, 24 bp, Artificial sequence
  Synthetic oligonucleotide
  misc_feature (1)..(2): where the internucleotide linkage is a phosphorothioate internucleotide linkage
  misc_feature (3)..(5): where the internucleotide linkages are phosphorothioate internucleotide linkages
  misc_feature (6)..(9): where the internucleotide linkages are phosphorothioate internucleotide linkages
  misc_feature (10)..(13): where the internucleotide linkages are phosphorothioate internucleotide linkages
  misc_feature (14)..(21): where the internucleotide linkages are phosphorothioate internucleotide linkages
  misc_feature (22)..(24): where the internucleotide linkages are phosphorothioate internucleotide linkages
tcgtcgttttgacgttttgtcgtt

SEQ ID NO: 33 - DNA, 24 bp, Artificial sequence
  Synthetic oligonucleotide
  misc_feature (1)..(2): wherein the internucleotide linkage is a phosphorothioate linkage
  misc_feature (3)..(5): wherein the internucleotide linkages are phosphorothioate linkages
  misc_feature (6)..(9): wherein the internucleotide linkages are phosphorothioate linkages
  misc_feature (10)..(13): wherein the internucleotide linkages are phosphorothioate linkages
  misc_feature (14)..(17): wherein the internucleotide linkages are phosphorothioate linkages
  misc_feature (18)..(21): wherein the internucleotide linkages are phosphorothioate linkages
  misc_feature (22)..(24): wherein the internucleotide linkages are phosphorothioate linkages
tcgtcgttttgacgttttgtcgtt

SEQ ID NO: 34 - DNA, 24 bp, Artificial sequence
  Synthetic oligonucleotide
  misc_feature (1)..(2): wherein the internucleotide linkage is a phosphorothioate linkage
  misc_feature (3)..(5): wherein the internucleotide linkages are phosphorothioate linkages
  misc_feature (6)..(7): wherein the internucleotide linkage is a phosphorothioate linkage
  misc_feature (8)..(9): wherein the internucleotide linkage is a phosphorothioate linkage
  misc_feature (10)..(11): wherein the internucleotide linkage is a phosphorothioate linkage
  misc_feature (12)..(13): wherein the internucleotide linkage is a phosphorothioate linkage
  misc_feature (14)..(15): wherein the internucleotide linkage is a phosphorothioate linkage
  misc_feature (16)..(17): wherein the internucleotide linkage is a phosphorothioate linkage
  misc_feature (18)..(19): wherein the internucleotide linkage is a phosphorothioate linkage
  misc_feature (20)..(21): wherein the internucleotide linkage is a phosphorothioate linkage
  misc_feature (22)..(24): wherein the internucleotide linkages are phosphorothioate linkages
tcgtcgttttgacgttttgtcgtt

SEQ ID NO: 35 - DNA, 24 bp, Artificial sequence
  Synthetic oligonucleotide
tcgtcgttttgacgttttgtcgtt

SEQ ID NO: 36 - DNA, 22 bp, Artificial sequence
  Synthetic oligonucleotide
  misc_feature (1)..(3): wherein the internucleotide linkages are phosphorothioate linkages
  misc_feature (4)..(11): wherein the internucleotide linkages are phosphorothioate linkages
  misc_feature (12)..(19): wherein the internucleotide linkages are phosphorothioate linkages
  misc_feature (20)..(22): wherein the internucleotide linkages are phosphorothioate linkages
gtcgttttgacgttttgtcgtt

SEQ ID NO: 37 - DNA, 21 bp, Artificial sequence
  Synthetic oligonucleotide
  misc_feature (1)..(2): wherein the internucleotide linkage is a phosphorothioate linkage
  misc_feature (3)..(5): wherein the internucleotide linkages are phosphorothioate linkages
  misc_feature (6)..(13): wherein the internucleotide linkages are phosphorothioate linkages
  misc_feature (14)..(21): wherein the internucleotide linkages are phosphorothioate linkages
tcgtcgttttgacgttttgtc

SEQ ID NO: 38 - DNA, 13 bp, Artificial sequence
  Synthetic oligonucleotide
  misc_feature (1)..(2): wherein the internucleotide linkage is a phosphorothioate linkage
  misc_feature (3)..(5): wherein the internucleotide linkage is a phosphorothioate linkage
  misc_feature (6)..(13): wherein the internucleotide linkage is a phosphorothioate linkage
tcgtcgttttgac

SEQ ID NO: 39 - DNA, 16 bp, Artificial sequence
  Synthetic oligonucleotide
  misc_feature (1)..(8): wherein the internucleotide linkages are phosphorothioate linkages
  misc_feature (9)..(16): wherein the internucleotide linkages are phosphorothioate linkages
gttttgacgttttgtc

SEQ ID NO: 40 - DNA, 19 bp, Artificial sequence
  Synthetic oligonucleotide
  misc_feature (1)..(8): wherein the internucleotide linkages are phosphorothioate linkages
  misc_feature (9)..(16): wherein the internucleotide linkages are phosphorothioate linkages
  misc_feature (17)..(19): wherein the internucleotide linkages are phosphorothioate linkages
gttttgacgttttgtcgtt

SEQ ID NO: 41 - DNA, 14 bp, Artificial sequence
  Synthetic oligonucleotide
  misc_feature (1)..(3): wherein the internucleotide linkages are phosphorothioate linkages
  misc_feature (4)..(11): wherein the internucleotide linkages are phosphorothioate linkages
  misc_feature (12)..(14): wherein the internucleotide linkages are phosphorothioate linkages
gtcgttttgacgtt

SEQ ID NO: 42 - DNA, 18 bp, Artificial sequence
  Synthetic oligonucleotide
  misc_feature (1)..(2): wherein the internucleotide linkage is a phosphorothioate linkage
  misc_feature (3)..(10): wherein the internucleotide linkages are phosphorothioate linkages
  misc_feature (11)..(18): wherein the internucleotide linkages are phosphorothioate linkages
tcgttttgacgttttgtc

SEQ ID NO: 43 - DNA, 19 bp, Artificial sequence
  Synthetic oligonucleotide
  misc_feature (1)..(3): wherein the internucleotide linkages are phosphorothioate linkages
  misc_feature (4)..(11): wherein the internucleotide linkages are phosphorothioate linkages
  misc_feature (12)..(19): wherein the internucleotide linkages are phosphorothioate linkages
gtcgttttgacgttttgtc

SEQ ID NO: 44 - DNA, 11 bp, Artificial sequence
  Synthetic oligonucleotide
  misc_feature (1)..(3): wherein the internucleotide linkages are phosphorothioate linkages
  misc_feature (4)..(11): wherein the internucleotide linkages are phosphorothioate linkages
gtcgttttgac

SEQ ID NO: 45 - DNA, 23 bp, Artificial sequence
  Synthetic oligonucleotide
  misc_feature (2)..(4): wherein the internucleotide linkages are phosphorothioate linkages
  misc_feature (5)..(12): wherein the internucleotide linkages are phosphorothioate linkages
  misc_feature (13)..(20): wherein the internucleotide linkages are phosphorothioate linkages
  misc_feature (21)..(23): wherein the internucleotide linkages are phosphorothioate linkages
cgtcgttttgacgttttgtcgtt

SEQ ID NO: 46 - DNA, 23 bp, Artificial sequence
  Synthetic oligonucleotide
  misc_feature (1)..(11): wherein the internucleotide linkages are phosphorothioate linkages
  misc_feature (14)..(23): wherein the internucleotide linkages are phosphorothioate linkages
tcgatcgtttttcgtgcgttttt

SEQ ID NO: 47 - DNA, 20 bp, Artificial sequence
  Synthetic oligonucleotide
  misc_feature (1)..(7): wherein the internucleotide linkages are phosphorothioate linkages
  misc_feature (8)..(15): wherein the internucleotide linkages are phosphorothioate linkages
  misc_feature (16)..(20): wherein the internucleotide linkages are phosphorothioate linkages
tcgcgacgttcgcgcgcgcg

SEQ ID NO: 48 - DNA, 19 bp, Artificial sequence
  Synthetic oligonucleotide
  misc_feature (1)..(2): wherein the internucleotide linkage is a phosphorothioate linkage
  misc_feature (3)..(6): wherein the internucleotide linkages are phosphorothioate linkages
  misc_feature (7)..(10): wherein the internucleotide linkages are phosphorothioate linkages
  misc_feature (11)..(19): wherein the internucleotide linkages are phosphorothioate linkages
tcggacgttcggcgcgccg

SEQ ID NO: 49 - DNA, 12 bp, Artificial sequence
  Synthetic oligonucleotide
cgacgttcgtcg

SEQ ID NO: 50 - DNA, 13 bp, Artificial sequence
  Synthetic oligonucleotide
cggcgccgtgccg

SEQ ID NO: 51 - DNA, 12 bp, Artificial sequence
  Synthetic oligonucleotide
ccccccgggggg

SEQ ID NO: 52 - DNA, 12 bp, Artificial sequence
  Synthetic oligonucleotide
ggggggcccccc

SEQ ID NO: 53 - DNA, 10 bp, Artificial sequence
  Synthetic oligonucleotide
cccccggggg

SEQ ID NO: 54 - DNA, 10 bp, Artificial sequence
  Synthetic oligonucleotide
gggggccccc

SEQ ID NO: 55 - DNA, 23 bp, Artificial sequence
  Synthetic oligonucleotide
  misc_feature (1)..(2): wherein the internucleotide linkage is a phosphorothioate linkage
  misc_feature (3)..(5): wherein the internucleotide linkages are phosphorothioate linkages
  misc_feature (6)..(8): wherein the internucleotide linkages are phosphorothioate linkages
  misc_feature (9)..(12): wherein the internucleotide linkages are phosphorothioate linkages
  misc_feature (13)..(17): wherein the internucleotide linkages are phosphorothioate linkages
  misc_feature (18)..(23): wherein the internucleotide linkages are phosphorothioate linkages
tgctcgtcgttcggcgcgcgccg

SEQ ID NO: 56 - DNA, 34 bp, Artificial sequence
  Synthetic oligonucleotide
  misc_feature (3)..(28): where n is any nucleotide and where any one or more n may be absent
  misc_feature (29)..(30): where n is any nucleotide
  misc_feature (33)..(34): where n is any nucleotide
tcnnnnnnnnnnnnnnnnnnnnnnnnntnncgnn